(19) United States
(12) Patent Application Publication
(10) Pub. No.: US 2003/0104404 A1
(43) Pub. Date: Jun. 5, 2003

(54) Genetic markers for autoimmune disorder

(76) Inventor: Carol A. Wise, Dallas, TX (US)

Correspondence Address: Margaret J. Sampson, VINSON & ELKINS LLP, 2300 First City Tower, 1001 Fannin, Austin, TX, 77002-6760, US

(21) Appl. No.: 10/067,076

(22) Filed: Feb. 4, 2002

Related U.S. Application Data
(63) Continuation-in-part of application No. 09/710,693, filed on Nov. 8, 2000.
(60) Provisional application No. 60/287,893, filed on May 1, 2001.

(51) Int. Cl. C12Q 1/68; G01N 33/567

(52) U.S. Cl. 435/6; 435/7.21

(57) ABSTRACT

Mutations in alleles of the gene encoding the CD2BP1 protein are identified as a causative factor in PAPA syndrome. Methods of diagnosing PAPA Syndrome and methods of screening for effectors of mutated protein binding and function are disclosed.

### STATEMENT REGARDING FEDERALLY SPONSORED RESEARCH OR DEVELOPMENT

Not applicable.

### REFERENCE TO A “Microfiche Appendix”

Not applicable.

### BACKGROUND OF THE INVENTION

1. Field of the Invention

The present invention is related to the field of inherited immunological disorders and to the discovery of genetic mutations as a causative factor in a disorder. The invention also relates to genetic screening for inherited disease, and in particular screening for markers for PAPA syndrome.

2. Description of Related Art

Juvenile idiopathic arthritis (JIA), also known as juvenile rheumatoid arthritis (JRA), is an autoimmune disorder comprising a heterogeneous collection of chronic arthritis of childhood (Fink et al., 1995, Pediatr Clin North Am 42:1155). This disease is common and affects 1-3 children per 1000. The pathogenesis of JIA as a whole is poorly understood, although genetics has been proposed to play a causative role. (Murray et al., 1997 Arch Dis Child 77:530; Moroldo et al., 1998, Arthritis Rheum 41:1620; Rossen et al., 1980, J Clin Invest 65:629; Rossen et al., 1982, Human Immunol 4:183). For pauciarticular onset JIA, in which the arthritis is limited to 4 or fewer joints during the first 6 months of disease, both family and population-based studies have revealed linkage and association with specific HLA alleles and disease. Associations with other immune complex molecules are also described (Heward and Gough, 1997, Clin Sci 93:479). There is, in addition, a striking gender effect for pauci-onset, with a ratio of approximately 8 to 1 girls to boys. Taken altogether the evidence for genetic predisposition to pauci-onset JIA is strong. However, a single causative gene has yet to be identified.

JIA is similar to other autoimmune disorders in that each appears to arise from complex genetic and environmental interactions (Heward and Gough, 1997; Vyse and Todd, 1996, Cell 85:311). Delineation of these factors and their mechanisms of action is ultimately critical to an understanding of the causes of the destruction of the body's own tissues. One approach to the genetic dissection of these complex disorders is positional cloning of predisposing genes within kindreds who demonstrate a simpler inheritance pattern, a strategy which has been successful in mapping and cloning genes involved in common diseases such as psoriasis (Tomfohrde et al., 1994, Science 264:1141), familial breast cancer (Hall, J M et al., 1990, Science 250:1684) and early onset Alzheimer's disease (St. George-Hyslop et al., 1992, Nat Genet 2:330).

The present inventor has described a new disorder that occurs in a subset of patients diagnosed as having juvenile idiopathic arthritis. This disorder has been termed “familial recurrent arthritis” or “FRA,” and was identified in a single large family originally carrying the diagnosis of JIA. FRA differs from “classic” JIA in several ways, but most notably in its striking pattern of dominant inheritance, in the pyogenic component of the joint swelling, and in the association with cutaneous findings. A genome wide linkage scan identified a 20 cM region within 15q22-24 most likely to harbor the predisposing gene.

Another disorder with striking similarity to FRA has been reported (Lindor et al., 1997, Mayo Clin Proc 72:611). The authors reported a single extended family with autosomal dominant transmission of a disorder characterized by pyogenic sterile arthritis, pyoderma gangrenosum, and severe cystic acne, which they referred to as “PAPA syndrome.” Like FRA, PAPA syndrome patients presented with acute inflammations which responded to steroid treatment, and laboratory findings were negative. Nine out of ten affected individuals in the PAPA syndrome family were reported to have arthritis in one to three joints, with age of onset varying from one to 16 years of age. Dermatological manifestations were variously found including pyoderma gangrenosum and severe cystic acne with onset at 11 years of age. The PAPA family was also reported to be karyotypically normal, and preliminary genotyping detected no linkage to the HLA region. From this, and given the recent onset of cystic acne in the proband FRA1-1, it is concluded that FRA and PAPA syndrome are likely the same disorder.

A further disorder has been described as “streaking leukocyte factor” (Jacobs and Goetzl, 1975, Pediatrics 56:570). The description of this disease appears very similar, if not identical to FRA and PAPA syndrome, as the patient described had a history of sterile pyarthrosis and cutaneous inflammation and ulceration since the second year of life, with no increased incidence of infections of any kind. Family history of the disorder, however, was not reported. Interestingly the authors reported partial purification of a serum factor (MW 160 kd) which enhanced the random migration of purified normal human neutrophils or mononuclear leukocytes but did not appear to affect chemotaxis.

There is still a need, however, to identify and isolate the specific gene or genes that are involved in PAPA syndrome, familial recurrent arthritis (FRA), and related disorders in order to provide means of diagnosis and management of the disorders, and to provide insights into the pathogenesis of the autoimmune joint destruction that is symptomatic of these and other autoimmune inflammatory conditions.

### BRIEF SUMMARY OF THE INVENTION

The present disclosure includes the identification of a gene encoding the CD2 binding protein, CD2BP1, as the inherited factor associated with familial recurrent arthritis (FRA), as well as PAPA syndrome. Co-segregating disease-causing mutations in the CD2BP1 gene have been found in families with this disorder, thus confirming that FRA and PAPA syndrome are the same disease. FRA and PAPA syndrome will be referred to herein collectively as “PAPA syndrome.” Certain preferred embodiments of the invention also include isolated nucleic acid molecules that encode mutations in the gene encoding CD2BP1, and particularly isolated genes that encode mutations in the region of the protein from amino acid 122 to 288. This is the region that is similar in sequence to the yeast CDC15 gene. More specifically, the gene allele associated with PAPA syndrome may contain mutations at the amino acid 230 or 250 positions, preferably A230T and/or E250Q. These mutations or others in the CD2BP1 gene may be detected as an aspect of a method of diagnosing PAPA syndrome in a subject, or in genetic screening of gametes, for example.

Preferred embodiments of the present disclosure include methods of diagnosing PAPA syndrome in a subject by detecting a mutation in a gene allele of a subject, wherein the gene allele encodes CD2BP1. In a preferred embodiment, the mutation in the gene allele of CD2BP1 is within the region of the gene that encodes from amino acid 122 to 288, inclusive in CD2BP1. In other preferred embodiments, the mutation includes a G748C transversion in a CD2BP1 gene, or a G688A transition in a CD2BP1 gene. In another preferred embodiment, the detection of the mutation is by denaturing HPLC or by DNA sequence analysis.

Another preferred embodiment of the present disclosure includes methods of diagnosing PAPA syndrome in a subject by identifying a single nucleotide polymorphisms (SNP) in the CD2BP1 gene by (a) obtaining a sample of nucleic acid from the subject; and (b) determining the identity of one or more SNPs in the CD2BP1 gene, wherein the SNPs are located at nucleotides 688 and 748. In a preferred embodiment, the SNP is located at nucleotide 688 and/or nucleotide 748 of the CD2BP1 gene. In another preferred embodiment, the subject is a human. In preferred embodiments, a SNP may be identified in a sample of nucleic acid from the subject, for example DNA or RNA, by a number of methods well known to those of skill in the art, including but not limited to DNA sequencing, DNA amplification, Oligonucleotide Ligation Assay (OLA), Doublecode OLA, Single Base Extension Assay, allele specific primer extension, or mismatch hybridization. Preferably, the identity of one or more SNPs in the CD2BP1 gene is determined by amplifying at least a portion of a nucleic acid molecule encoding the CD2BP1 gene, or by sequencing at least a portion of a nucleic acid molecule encoding the CD2BP1 gene.

Another aspect of the present disclosure includes methods of screening for agents that modify an immune response in cells and/or in organisms that also express a CD2BP1 gene product with a mutation associated with PAPA syndrome. In a preferred embodiment, the mutation is in the region of CD2BP1 bounded by amino acids 122 to 288 inclusive. In other preferred embodiments, the mutation includes a G748C transversion in a CD2BP1 gene as numbered in SEQ ID NO:21, or a G688A transition in a CD2BP1 gene as numbered in SEQ ID NO:19. In another preferred embodiment, the one or more mutations in CD2BP1 are in amino acid 250 and/or amino acid 230, including but not limited to an E250Q mutation, an A230T mutation, or both E250Q and A230T mutations. As used herein, a term such as E250Q denotes a mutation in the protein sequence of CD2BP1 at the position of amino acid 250. The first letter is the one letter designation of the naturally occurring amino acid, glutamic acid (E, Glu) and the last letter is the one letter designation of the amino acid that is substituted, which is glutamine (Q, Gln). As used herein, A230T denotes a mutation in the protein sequence of CD2BP1 at the position of amino acid 230; the naturally occurring amino acid at this position is alanine (A, Ala), and the mutant amino acid is threonine (T, Thr). Such assays typically include contacting cells or tissues with an agent suspected of modifying an immune response, measuring an indicator of immune response, and comparing that measurement to the same immune response indicator in a control cell or tissue under comparable conditions in the absence of the agent. A difference in the measured response in comparison to the measured control is indicative of an agent that modifies an immune response. It is understood that an agent that modifies an immune response may inhibit or enhance the immune response. In other preferred embodiments the indicator is T-cell rosetting, altered motility of immune cells, calcium flux, I1-2 production, cytolytic activity, CD2 triggered adhesion involving CD58, or integrin-mediated adhesion activated through CD2 or a CD15 carrier; preferably the CD15 carrier is CD66a.

Preferred cells for use in the described assays would include immune cells such as T-cells, monocytes, neutrophils, or NK cells for example, and would also include cells or tissues that include cells that express CD2 or even CD15 expressing cells, either naturally occurring cells, or cells that have been engineered to express recombinant CD2 or CD15.

One may use any immune response assay known in the art. Such assays would include, but are not limited to, T-cell rosetting, altered motility of immune cells, calcium flux, I1-2 production, and cytolytic activity.

Another aspect of the disclosure is methods of screening for an agent that modifies an interaction of the CD2BP1 gene or gene products with a binding partner of CD2BP1, wherein the CD2BP1 has a mutation associated with PAPA syndrome. In a preferred embodiment, the mutation is in the region of CD2BP1 bounded by amino acids 122 to 288 inclusive. In another preferred embodiment, the one or more mutations in CD2BP1 are in amino acid 250 and/or amino acid 230, including but not limited to an E250Q mutation, an A230T mutation, or both E250Q and A230T mutations. In another preferred embodiment, binding assays may include determining the binding of the binding partner of CD2BP1, for example the cytoplasmic portion of CD2, and the mutant CD2BP1 in the presence of an agent suspected of altering the binding interaction of the binding partner and CD2BP1, and comparing the binding in the presence of the agent to the binding interaction in the absence of the agent, wherein a difference in the binding interaction is indicative of an effector of the binding partner to the mutant CD2BP1. Such assays may also be used to screen for agents that modify the interaction of a mutant CD2BP1 with PTP PEST (Li, et al., EMBO 17(24):7320-7336, 1998); the interaction with the human homolog of PTP HSCF (Spencer et al., J. Cell. Biol. 138(4):845-60 (1997); Dowbenko et al., J Biol. Chem. 273(2):989-96, 1998); the interaction with the human homolog of the Wiskott-Aldrich Syndrome Protein (WASP) (Wu et al., J. Biol. Chem., 273(10), 5765-5770 1998); the interaction with pyrin (Shoham et al, The FMF Protein, Pyrin, Specifically Interacts with the SH3 Domain of the Cytoskeletal Protein PSTPIP1, The American Society of Human Genetics, 51st Annual Meeting, Oct. 12-16, 2001, Abstract No. 1100); the interaction with c-Abl (Cong et al., Mol. Cell, 6(6):1413-1423, 2000); or the interaction with a CD15 carrier, for example the human homolog of CD66a (Warren et al., J. Immunology, 156: 2876-2873, 1996; Stocks and Kerr, Biochem J., 288, 23-27, 1992; Stocks, et al., Biochem J. 268, 275-280, 1990; Kammerer et al, Eur. J. Immunology, 28(11):3664-3674, 1998; Lucka et al., FEBS Letters, 438:37-40, 1998).

Binding assays may be any known in the art and would include immunoprecipitation or Western blot assays with monoclonal antibodies to any of the binding partners, or an assay that includes a fusion of a protein such as CD2BP1 to an immunological marker such as GST. Other assays would include the yeast two-hybrid assay as described in Li et al. (EMBO 17(24):7320-7336, 1998) (incorporated herein by reference).

Another aspect of the present disclosure includes methods of screening for agents that modify or prevent apoptosis in cells and/or in organisms that also express a CD2BP1 gene product with a mutation associated with PAPA syndrome. Apoptotic activity is the ability either alone, or in combination with another molecule, to produce cell death accompanied by at least one of the morphological or biochemical alterations characteristic of apoptosis. For example, the classical biochemical alteration characteristic of apoptosis is the appearance of oligonucleosome-sized fragments of DNA, which produce a “ladder” upon agarose gel electrophoresis. In a preferred embodiment, the mutation is in the region of CD2BP1 bounded by amino acids 122 to 288 inclusive. In another preferred embodiment, the one or more mutations in CD2BP1 are in amino acid 250 and/or amino acid 230, including but not limited to an E250Q mutation, an A230T mutation, or both E250Q and A230T mutations. In another preferred embodiment the apoptosis is pyrin-mediated apoptosis. Such assays typically include contacting cells or tissues with an agent suspected of modifying or preventing apoptotic activity, measuring an indicator of apoptotic activity, and comparing that measurement to the same apoptotic activity in a control cell or tissue under comparable conditions in the absence of the agent. A difference in the measured apoptotic activity in comparison to the measured control is indicative of an agent that modifies or prevents apoptosis. It is understood that an agent that modifies apoptosis may increase or decrease apoptosis. In other preferred embodiments the indicators of apoptotic activity include but are not limited to light microscopy for determining the presence of one or more morphological characteristics of apoptosis, such as condensed or rounded morphology, shrinking and blebbing of the cytoplasm, preservation of structure of cellular organelles including mitochondria, and condensation and margination of chromatin. Other methods and assays for determining apoptotic activity are well known in the art, and one skilled in the art understands the assays for apoptotic or anti-apoptotic activity that can be performed using routine methodology.

Embodiments of the disclosure also include isolated nucleic acid molecules that encode mutations in the gene encoding CD2BP1 associated with PAPA syndrome, and particularly isolated genes that encode an amino acid sequence including the sequence of SEQ ID NO:20 or SEQ ID NO:22, together with conservative amino acid substitutions, and a composition including a partially purified protein encoded by the nucleic acid molecules. Embodiments of the disclosure also include isolated nucleic acid molecules that include the nucleic acid sequence of SEQ ID NO:19 or SEQ ID NO:21, including expression vectors and host cells transfected with such vectors. In other preferred embodiments, an expression construct includes a nucleic acid molecule that encodes the amino acid sequence of SEQ ID NO:20 or SEQ ID NO:22, and conservative amino acid substitutions thereof, operably linked to an expression control sequence. Preferably the expression construct is a plasmid expression vector or a viral expression vector. In another preferred embodiment, a host cell is transformed or transfected with the expression vector encoding a mutant CD2BP1, wherein the host cell is a bacterial, mammalian, or a human cell.

Another preferred embodiment of the present disclosure is an isolated nucleic acid molecule that includes a SNP in the CD2BP1 gene that is associated with PAPA syndrome. Preferably, the isolated nucleic acid molecule includes contiguous nucleotides of the CD2BP1 gene, as disclosed in SEQ ID NO:18. In preferred embodiments, the isolated nucleic acid molecule with a SNP in the CD2BP1 gene can be about 15, 16, 17, 18, 19, 20, 21, 22, 23, 24, 24, 26, 27, 28, 29, 30, 40, 50, 100, 200, 300, 400, 500, 600, 700, 800, 900, and 1000 or more contiguous nucleotides in length. In a preferred embodiment, the isolated nucleic acid molecule is at least about 20 contiguous nucleotides of SEQ ID NO:18, and includes one or both of the following: nucleotide 688 wherein the G is replaced by an A, or nucleotide 748 wherein the G is replace by a C. In another preferred embodiment, the nucleotide corresponding to nucleotide 688 or 748 of SEQ ID NO:18 is located at the 3′ end of the isolated nucleic acid molecule. In yet another preferred embodiment, the nucleotide corresponding to nucleotide 688 or 748 of SEQ ID NO:18 is located at the 5′ end of the isolated nucleic acid molecule. A preferred embodiment of the present disclosure is an isolated nucleic acid molecule that is the complement of the nucleic acid molecule that includes contiguous nucleotides of SEQ ID NO:18, and includes one or both of the following: nucleotide 688 wherein the G is replaced by an A, or nucleotide 748 wherein the G is replace by a C.

An additional preferred embodiment of the present disclosure is an array of nucleic acid molecules attached to a solid support, the array comprising an oligonucleotide that will hybridize to a nucleic acid molecule that includes contiguous nucleotides of SEQ ID NO:18, and includes one or both of the following: nucleotide 688 wherein the G is replaced by an A, or nucleotide 748 wherein the G is replace by a C, under conditions in which the oligonucleotide will not substantially hybridize to a nucleic acid molecule consisting of SEQ ID NO:18.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Alignment of the amino acid sequences of human CD2BP1 and murine PSTPIP. The amino acid sequence of CD2BP1 is designated in FIG. 1 as SEQ ID NO:1; the N-terminal amino acid sequence of PSTPIP from 1-323 is designated in FIG. 1 as SEQ ID NO:2; and the C-terminal amino acid sequence of PSTPIP from 324-416 is designated in FIG. 1 as SEQ ID NO:3. Sequences exhibiting high homology to yeast cdc15 include the region from amino acid 122-288. The sequences with high homology to a typical SH3 domain are amino acids 360-417. The potential PEST regions are underlined.

FIG. 2. Structure of the CD2BP1 gene. Predicted exons are shown by alternating gray and black shading, and are numbered below each line, with the exception of exon 6, which is numbered above the line. Primer sequences used for PCR amplification in Example 2 are underlined; primer names are shown in bold print above the sequence for forward primer and below the sequence for reverse primers. The G688A and G748C mutations are capitalized. The sequence shown in FIG. 2 is SEQ ID NO:28.

### DETAILED DESCRIPTION OF THE INVENTION

The present disclosure arises from the discovery that CD2BP1, an adaptor protein, is mutated in individuals with PAPA syndrome. As used herein, PAPA syndrome refers collectively to familial recurrent arthritis (FRA) and PAPA syndrome. This finding identifies CD2BP1 as central to a biochemical pathway which, when altered, can trigger inappropriate immune or autoimmune responses. The discovery of mutations in the CD2BP1 gene also provide for methods of screening for PAPA syndrome for the purposes of diagnosing the disease and genetic counseling. In addition, the mutated protein may be used for high throughput assays to discover complementary mutations in the biochemical pathway, and/or for the discovery of therapeutic agents useful for the treatment or management of PAPA syndrome, FRA, or other immune disorders.

An aspect of the present disclosure is the binding interaction of the mutant CD2BP1 to the known interactors, PTP PEST (protein tyrosine phosphatase, proline-glutamate-serine-threonine rich region), CD2, and pyrin, the levels of phosphorylation of the mutant protein, and the effects of the binding and phosphorylation state on the pathology of immune disorders. In addition, an aspect of the present invention is the binding interaction of the mutant CD2BP1 with interactors identified in the murine system, PTP HSCF, WASP, and c-Abl. Another aspect of the present invention is the potential binding interaction of the mutant CD2BP1 and (human) CD15 carriers, in particular, CD66a. In the murine system, the murine homolog of CD2BP1, proline, serine, threonine phosphatase interacting protein (PSTPIP), has been demonstrated to bind to another protein tyrosine phosphatase, PTP HSCF (protein tyrosine phosphatase hematopoietic stem cell fraction). This can be achieved with a portion of the PTP HSCF protein containing amino acids 1-264. A W232A mutation of PSTPIP results in complete loss of binding to PTP HSCF in vitro, as well as loss of association of the two proteins in co-transfected COS cells. Little or no change in binding activity was seen in alanine scanning mutants of various positions throughout the N-terminal coiled-coil domain of PSTPIP, suggesting that the region critical for PTP HSCF binding is within amino acids 232-264 of the PSTPIP protein (Dowbenko et al., 1998, Journal of Biological Chemistry 273:989), which would include (in the homolog) an amino acid reported herein to be associated with PAPA Syndrome (E250Q). Furthermore, this region may be influenced by a second mutation disclosed herein, A230T. The murine homolog PSTPIP also has been demonstrated to interact through its SH3 domain with the murine homolog of the Wiskott Aldrich Syndrome Protein (WASP), an event which appears to be regulated by tyrosine phosphorylation of PSTPIP. CD15 carriers are potential interactors with CD2BP1; CD15 levels measured on neutrophils of FRA patients are approximately 10-15% of normal. In the previous case report of what was described as “streaking leukocyte factor” (Jacobs and Goetzl, 1975, Pediatrics 56:570), the authors reported partial purification of a serum factor (MW 160 kd) which enhanced the random migration of purified normal human neutrophils or mononuclear leukocytes but did not appear to affect chemotaxis. This protein may be the potential CD2BP1 interactor CD66a, the major carrier of CD15 on neutrophils.

The A230T and E250Q mutations alter the binding of human PTP HSCF, and are subsequently contemplated to change the degree of phosphorylation of the CD2BP1 protein. Additionally, this mutation may alter interaction with an as yet unidentified protein that is expressed in a cell-specific manner. This is a plausible scenario given that the E250Q or A230T mutations result in a recurring autoimmune inflammation limited to a few physiological sites, rather than a global compromise of immune function.

The present disclosure therefore, encompasses:

(a) DNA vectors that contain a mutant CD2BP1 encoding sequence as described herein and/or their complements (i.e., antisense);

(b) DNA expression vectors that contain any of the foregoing mutant CD2BP1 coding sequences operatively associated with a regulatory element that directs the expression of the coding sequences;

(c) genetically engineered host cells that contain any of the foregoing mutant CD2BP1 coding sequences operatively associated with a regulatory element that directs the expression of the coding sequences in the host cell; and

(d) genetically engineered organisms that contain any of the foregoing mutant CD2BP1 coding sequences operatively associated with a regulatory element that directs the expression of the coding sequences in one or more cells or tissues of the genetically modified organism.

As used herein, regulatory elements include but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast alpha-mating factors.

The invention further includes fragments of any of the DNA sequences disclosed herein, in particular those fragments that include the coding region of amino acid 230 and/or 250 of the CD2BP1 gene and more particularly those that include a missense mutation at amino acid 230 and/or 250, and even more particularly those that include a G to C transversion at nucleotide 748 as numbered in SEQ ID NO:19, and/or a G to A transition at nucleotide 688 as numbered in SEQ ID NO:21. In one embodiment, the CD2BP1 gene sequences of the invention are human gene sequences, with homologous mammalian gene sequences with the analogous mutations also being included, in particular a sequence encoding a mutation at amino acid 230 and/or 250 in the murine PSTPIP protein.

In the practice of the present invention, mutant CD2BP1 gene products, or peptide fragments thereof, can be prepared for a variety of uses. For example, such gene products, or peptide fragments thereof, can be used for the generation of antibodies, in diagnostic assays, or for the identification of other cellular or extracellular gene products involved in the regulation of a CD2BP1 mediated disorder, such as PAPA syndrome. The amino acid sequences of SEQ ID NO:20 and SEQ ID NO:22 represent mutant CD2BP1 gene products. The CD2BP1 gene product includes both wild-type and mutant CD2BP1 gene products. A mutant CD2BP1 gene product is sometimes referred to herein as a “mutant CD2BP1 protein,” or a “mutant CD2BP1,” and includes those gene products encoded by the CD2BP1 gene sequences described above. In addition, CD2BP1 gene products may include proteins that represent functionally equivalent gene products. Such an equivalent CD2BP1 gene product may also contain deletions, including internal deletions; additions, including additions yielding fusion proteins; or substitutions of amino acid residues within and/or adjacent to the amino acid sequence encoded by the CD2BP1 gene sequences described, above, but that result in a “silent” change, in that the change produces a functionally equivalent mutant CD2BP1 gene product. Amino acid substitutions may be conservative amino acid substitutions, which are made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Alternatively, where alteration of function is desired, deletions or non-conservative alterations can be engineered to produce altered CD2BP1 gene products. Such alterations can, for example, include the substitution of other amino acids at position 230 and/or 250 in the CD2BP1 protein, or the deletion or substitution of amino acids in the region of the protein involved in binding to PTP PEST, WASP, PTP HSCF, CD15 carriers, CD2, c-Abl, or pyrin, such as amino acids 122 to 288, or amino acids 232-264, or the SH3 region, amino acids 360-417 of CD2BP1. Alterations in the CD2BP1 protein may therefore be chosen to affect binding to the known or unknown cytoplasmic interactors as well as to soluble factors, ligands, or cell surface proteins or markers that interact with CD2 or CD2BP1 at the cell surface.

The CD2BP1 gene products, peptide fragments thereof and fusion proteins thereof, may be produced by recombinant DNA technology using techniques well known in the art. Thus, methods for preparing the CD2BP1 gene polypeptides, peptides, fusion peptide and fusion polypeptides of the invention by expressing nucleic acid containing CD2BP1 gene sequences are described herein. Methods that are well known to those skilled in the art can be used to construct expression vectors containing CD2BP1 gene product coding sequences and appropriate transcriptional and translational control signals. These methods include, for example, in vitro recombinant DNA techniques, synthetic techniques, and in vivo genetic recombination. See, for example, the techniques described in Sambrook, et al., 1989, Molecular Cloning, A Laboratory Manual, Second Edition, Cold Spring Harbor Press, N.Y; and Ausubel, et al., eds., 1989, Current Protocols in Molecular Biology, vol. I, Green Publishing Associates, Inc., and John Wiley & Sons, Inc., N.Y., at p. 2.10.3. Alternatively, RNA capable of encoding CD2BP1 gene product sequences may be chemically synthesized using, for example, synthesizers. See, for example, the techniques described in “Oligonucleotide Synthesis,” 1984, Gait, ed., IRL Press, Oxford.

A variety of host-expression vector systems may be utilized to express the CD2BP1 gene coding sequences of the invention. Such host-expression systems represent vehicles by which the coding sequences of interest may be produced and subsequently purified, but also represent cells that may, when transformed or transfected with the appropriate nucleotide coding sequences, exhibit the CD2BP1 gene product of the invention in situ. These include but are not limited to microorganisms such as bacteria (e.g., E. coli, B. subtilis) transformed with recombinant bacteriophage DNA, plasmid DNA or cosmid DNA expression vectors containing CD2BP1 gene product coding sequences; yeast (e.g., Saccharomyces, Pichia) transformed with recombinant yeast expression vectors containing the CD2BP1 gene product coding sequences; insect cell systems infected with recombinant virus expression vectors (e.g., baculovirus) containing the CD2BP1 gene product coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CD2BP1 gene product coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter, CMV promoter).

In bacterial systems, a number of expression vectors may be advantageously selected depending upon the use intended for the CD2BP1 gene product being expressed. For example, when a large quantity of such a protein is to be produced, for the generation of highly purified compositions of CD2BP1 protein or for raising antibodies to CD2BP1 protein, for example, vectors that direct the expression of high levels of fusion protein products that are readily purified may be desirable. Such vectors include, but are not limited, to the E. coli expression vector pUR278 (Ruther et al., 1983, EMBO J. 2, 1791), in which the CD2BP1 gene product coding sequence maybe ligated individually into the vector in frame with the lac Z coding region so that a fusion protein is produced; pIN vectors (Inouye and Inouye, 1985, Nucleic Acids Res. 13, 3101-3109; Van Heeke and Schuster, 1989, J. Biol. Chem. 264, 5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica, nuclear polyhedrosis virus (AcNPV) is used as a vector to express foreign genes. The virus grows in Spodoptera frugiperda cells. The CD2BP1 gene coding sequence may be cloned individually into non-essential regions (for example the polyhedrin gene) of the virus and placed under control of an ACNPV promoter (for example the polyhedrin promoter). Successful insertion of CD2BP1 gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells, in which the CD2BP1 gene product is expressed (e.g., see Smith, et al., 1983, J. Virol. 46, 584; Smith, U.S. Pat. No. 4,215,051).

In mammalian host cells, a number of viral-based expression systems may be utilized. In cases where an adenovirus is used as an expression vector, the CD2BP1 gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CD2BP1 gene product in infected hosts. (e.g., See Logan and Shenk, 1984, Proc. Natl. Acad. Sci. U.S.A. 81, 3655-3659). Specific initiation signals may also be required for efficient translation of inserted CD2BP1 gene product coding sequences. These signals include the ATG initiation codon and adjacent sequences. In cases where an entire CD2BP1 gene, including its own initiation codon and adjacent sequences, is inserted into the appropriate expression vector, no additional translational control signals may be needed. However, in cases where only a portion of the CD2BP1 gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner, et al., 1987, Methods in Enzymol. 153, 516-544).

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, and W138.

For long-term, high-yield production of recombinant proteins, stable expression is preferred. For example, cell lines that stably express the CD2BP1 gene product may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method may advantageously be used to engineer cell lines that express the CD2BP1 gene product. Such engineered cell lines may be particularly useful in screening and evaluation of compounds that affect the endogenous activity of the CD2BP1 gene product.

A number of selection systems may be used, including but not limited to the herpes simplex virus thymidine kinase (Wigler, et al., 1977, Cell 11, 223), hypoxanthine-guanine phosphoribosyltransferase (Szybalska and Szybalski, 1962, Proc. Natl. Acad. Sci. U.S.A. 48, 2026), and adenine phosphoribosyltransferase (Lowy, et al., 1980, Cell 22, 817) genes can be employed in tk-, hgprt-, or aprt-cells, respectively. Also, antimetabolite resistance can be used as the basis of selection for the following genes: dhfr, which confers resistance to methotrexate (Wigler, et al., 1980, Natl. Acad. Sci. U.S.A. 77, 3567; O'Hare, et al., 1981, Proc. Natl. Acad. Sci. U.S.A. 78, 1527); gpt, which confers resistance to mycophenolic acid (Mulligan and Berg, 1981, Proc. Natl. Acad. Sci. U.S.A. 78, 2072); neo, which confers resistance to the aminoglycoside G-418 (Colberre-Garapin, et al., 1981, J. Mol. Biol. 150, 1); and hygro, which confers resistance to hygromycin (Santerre, et al., 1984, Gene 30, 147).

Alternatively, any fusion protein may be readily purified by utilizing an antibody or ligand specific for the fusion protein being expressed. For example, a system described by Janknecht, et al. allows for the ready purification of non-denatured fusion proteins expressed in human cell lines (Janknecht, et al., 1991, Proc. Natl. Acad. Sci. U.S.A. 88, 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers. HIS-tagged fusion proteins may be produced from plasmid vectors as well.

Polymorphisms, for example SNPs, that are identified as being associated with PAPA syndrome can be screened using a variety of techniques well known to those of skill in the art. SNPs are stable nucleotide sequence variations at a specific location in the genome of different individuals. SNPs are of predictive value in identifying many genetic diseases, as well as phenotypic characteristics that may be desirable, which are often caused by a limited number of different mutations in a population. SNPs are found in both coding and non-coding regions of genomic DNA, and are found in large numbers throughout the human genome (Cooper et al., Hum Genet 69:201-205, 1985). Certain SNPs result in disease-causing mutations such as, for example, heritable breast cancer (Cannon-Albright and Skolnick, Semin Oncol 23:1-5, 1996). Current methods of screening for polymorphisms are known (see for example U.S. Pat. Nos. 6,221,592 and 5,679,524, incorporated herein by reference).

A SNP may be identified in the nucleic acid material of an organism by a number of methods well known to those of skill in the art, including but not limited to identifying the SNP by PCR™ or DNA amplification, Oligonucleotide Ligation Assay (OLA) (Landegren et al., Science 241:1077, 1988, incorporated herein by reference), Doublecode OLA (described in U.S. application Ser. No. 09/755,628, incorporated herein by reference), mismatch hybridization, mass spectrometry, Single Base Extension Assay, RFLP detection based on allele-specific restriction-endonuclease cleavage (Kan and Dozy, Lancet ii:910-912, 1978, incorporated herein by reference), hybridization with allele-specific oligonucleotide probes (Wallace et al., Nucl Acids Res 6:3543-3557, 1978, incorporated herein by reference), including immobilized oligonucleotides (Saiki et al., Proc Natl Acad Sci U.S.A. 86:6230-6234, 1989, incorporated herein by reference) or oligonucleotide arrays (Maskos and Southern, Nucl Acids Res 21:2269-2270, 1993, incorporated herein by reference), allele-specific PCR™ (Newton et al., Nucl Acids Res 17:2503-16, 1989, incorporated herein by reference), mismatch-repair detection (MRD) (Faham and Cox, Genome Res 5:474-482, 1995, incorporated herein by reference), binding of MutS protein (Wagner et al., Nucl Acids Res 23:3944-3948, 1995, incorporated herein by reference), single-strand-conformation-polymorphism detection (Orita et al., Genomics 5:874-879, 1983, incorporated herein by reference), RNAase cleavage at mismatched base-pairs (Myers et al., Science 230:1242, 1985, incorporated herein by reference), chemical (Cotton et al., Proc Natl Acad Sci U.S.A. 85:4397-4401, 1988, incorporated herein by reference) or enzymatic (Youil et al., Proc Natl Acad Sci U.S.A. 92:87-91, 1995, incorporated herein by reference) cleavage of heteroduplex DNA, methods based on allele specific primer extension (Syvanen et al., Genomics 8:684-692, 1990, incorporated herein by reference), genetic bit analysis (GBA) (Nikiforov et al., Nuci Acids Res 22:4167-4175, 1994, incorporated hereinby reference), and radioactive and/or fluorescent DNA sequencing using standard procedures well known in the art.

In the context of the present disclosure, it is also specifically contemplated that nucleic acid samples of subjects in a population, preferably humans, may be analyzed using DNA chips or microarrays in order to detect specific genetic sequences, including genetic polymorphisms or genetic variations, such as for example SNPs. In one embodiment, it is envisioned that genomic DNA will be amplified in order to produce a library of DNA sequences theoretically encompassing the entire genomic sequence. The amplified DNA products may then be passed over a DNA chip or microarray encompassing oligonucleotide or polynucleotide probes. The ability or inability of the amplified DNA to hybridize to the microarray or DNA chip will facilitate the characterization of the specific sequences and their polymorphisms present in the DNA sample, for example SNPs that are identified as being associated with PAPA syndrome.

The CD2BP1 gene products can also be expressed in transgenic animals. Animals of any species, including, but not limited to, mice, rats, rabbits, guinea pigs, pigs, micro-pigs, goats, sheep, and non-human primates, e.g., baboons, monkeys, and chimpanzees may be used to generate CD2BP1 transgenic animals. The term “transgenic,” as used herein, refers to animals expressing CD2BP1 gene sequences from a different species (e.g., mice expressing human CD2BP1 sequences), as well as animals that have been genetically engineered to overexpress endogenous (i.e., same species) CD2BP1 sequences or animals that have been genetically engineered to no longer express endogenous CD2BP1 gene sequences (i.e., “knock-out” animals), and their progeny. Transgenic expression of mutant CD2BP1 proteins as described herein provides, for example, an important animal model for the study of immune disorders including PAPA syndrome.

Any technique known in the art may be used to introduce a mutant CD2BP1 gene transgene into animals to produce the founder lines of transgenic animals. Such techniques include, but are not limited to pronuclear microinjection (Hoppe and Wagner, 1989, U.S. Pat. No. 4,873,191); retrovirus mediated gene transfer into germ lines (Van der Putten, et al., 1985, Proc. Natl. Acad. Sci., U.S.A. 82, 6148-6152); gene targeting in embryonic stem cells (Thompson, et al., 1989, Cell 56, 313-321); electroporation of embryos (Lo, 1983, Mol. Cell. Biol. 3, 1803-1814); and sperm-mediated gene transfer (Lavitrano et al., 1989, Cell 57, 717-723). (For a review of such techniques, see Gordon, 1989, Transgenic Animals, Intl. Rev. Cytol. 115; 171-229).

Any technique known in the art may be used to produce transgenic animal clones containing an CD2BP1 transgene, for example, nuclear transfer into enucleated oocytes of nuclei from cultured embryonic, fetal, or adult cells induced to quiescence (Campbell, et al., 1996, Nature 380, 64-66; Wilmut, et al., Nature 385, 810-813).

The present invention thus provides for transgenic animals that carry a mutant CD2BP1 transgene in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type, hematopoietic cells, for example, by following the teaching of Lasko et al. (Lasko, et al., 1992, Proc. Natl. Acad. Sci. U.S.A. 89, 6232-6236). The present invention also provides for transgenic animals that overexpress a wild-type CD2BP1 transgene in some or all of their cells. The regulatory sequences required for such a cell-type specific activation will depend upon the particular cell type of interest, and will be apparent to those of skill in the art. When it is desired that the CD2BP1 gene transgene be integrated into the chromosomal site of the endogenous CD2BP1 gene, gene targeting is preferred. Briefly, when such a technique is to be utilized, vectors containing some nucleotide sequences homologous to the endogenous CD2BP1 gene are designed for the purpose of integrating, via homologous recombination, with chromosomal sequences, into and disrupting the function of the nucleotide sequence of the endogenous CD2BP1 gene. The transgene may also be selectively introduced into a particular cell type, thus inactivating the endogenous CD2BP1 gene in only that cell type, by following, for example, the teaching of Gu, et al. (Gu, et al., 1994, Science 265, 103-106). The regulatory sequences required for such a cell-type specific inactivation will depend upon the particular cell type of interest, and will be apparent to those of skill in the art.

Once transgenic animals have been generated, the expression of the recombinant mutant CD2BP1 gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include but are not limited to Northern blot analysis of tissue samples obtained from the animal, in situ hybridization analysis, and RT-PCR (reverse transcriptase PCR). Samples of CD2BP1 gene-expressing tissue may also be evaluated immunocytochemically or by other in vitro techniques such as Western Blotting using antibodies specific for the CD2BP1 transgene product.

Also described herein are methods for the production of antibodies capable of specifically recognizing one or more mutant CD2BP1 gene product epitopes or epitopes of conserved variants or peptide fragments of the CD2BP1 gene products. Because a most useful antibody would be one that could distinguish the mutant CD2BP1 protein from the wild type, monoclonal antibodies are preferred. Such antibodies may be used, for example, in the detection of a CD2BP1 gene product in a biological sample and may, therefore, be utilized as part of a diagnostic or prognostic technique whereby patients may be tested for abnormal CD2BP1 gene products. Such antibodies may also be utilized in conjunction with, for example, compound screening schemes for the evaluation of the effect of test compounds on CD2BP1 gene product activity.

For the production of antibodies against a CD2BP1 gene product, various host animals may be immunized by injection with a mutant CD2BP1 gene product, or a portion thereof. Such host animals may include, but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler and Milstein, (1975, Nature 256, 495-497; and U.S. Pat. No. 4,376,110), the human B-cell hybridoma technique (Kosbor et al., 1983, Immunology Today 4,72; Cole et al., 1983, Proc. Natl. Acad. Sci. U.S.A. 80, 2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD, and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314,452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816397, which are incorporated herein by reference in their entirety).

Techniques have also been developed for the production of humanized antibodies. (See, e.g., Queen, U.S. Pat. No. 5,585,089, which is incorporated herein by reference in its entirety). An immunoglobuin light or heavy chain variable region consists of a “framework” region interrupted by three hypervariable regions, referred to as complementarity determining regions (CDRs). The extent of the framework region and CDRs have been precisely defined (see, “Sequences of Proteins of Immunological Interest,” Kabat, E. et al., U.S. Department of Health and Human Services (1983)). Briefly, humanized antibodies are antibody molecules from non-human species having one or more CDRs from the non-human species and a framework region from a human immunoglobulin molecule. Alternatively, techniques described for the production of single chain antibodies (U.S. Pat. No. 4,946,778; Bird, 1988, Science 242, 423-426; Huston, et al., 1988, Proc. Natl. Acad. Sci. U.S.A. 85, 5879-5883; and Ward, et al., 1989, Nature 334,544-546) can be adapted to produce single chain antibodies against CD2BP1 gene products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)2 fragments, which can be produced by pepsin digestion of the antibody molecule and the Fab fragments, which can be generated by reducing the disulfide bridges of the F(ab′)2 fragments. Alternatively, Fab expression libraries may be constructed (Huse, et al., 1989, Science, 246, 1275-1281) to allow rapid and easy identification of monoclonal Fab fragments with the desired specificity.

An aspect of the present disclosure is also various applications of CD2BP1 gene sequences, CD2BP1 gene products, including peptide fragments and fusion proteins thereof, and of antibodies directed against CD2BP1 gene products and peptide fragments thereof. Such applications include, for example, prognostic and diagnostic evaluation of a CD2BP1 mediated immune disorder such as PAPA syndrome, and the identification of subjects with a predisposition to such disorders. Additionally, such applications include methods for the identification of compounds that modulate the expression of the CD2BP1 gene and/or the synthesis or activity of the CD2BP1 gene product. Such compounds can include, for example, other cellular products that are involved in the immune response signaling processes.

A variety of methods can be employed to screen for the presence of CD2BP1 mutations, and in particular the PAPA-syndrome associated mutation described herein. Nucleic acid from any nucleated cell can be used as the starting point for such assay techniques, and may be isolated according to standard nucleic acid preparation procedures that are well known to those of skill in the art. CD2BP1 nucleic acid sequences may be used in hybridization or amplification assays of biological samples to detect the described mutation in the CD2BP1 gene structure. Such assays may include, but are not limited to, denaturing HPLC, Southern analyses, single-stranded conformational polymorphism analyses (SSCP), DNA sequencing and polymerase chain reaction (PCR) analyses. Diagnostic methods for the detection of the mutant CD2BP1 gene, in patient samples or other appropriate cell sources, may thus involve their amplification, e.g., by PCR (the experimental embodiment set forth in Mullis, 1987, U.S. Pat. No. 4,683,202), followed by the detection and/or nucleotide sequencing of the amplified molecules using standard techniques.

An aspect of the invention is also assays designed to identify compounds that bind to a mutant CD2BP1 gene product and intracellular proteins or portions of proteins that interact with a mutant CD2BP1 gene product. Such intracellular proteins or portions of proteins may be involved in the control and/or regulation of an immune response.

Compounds that may be so identified include, but are not limited to, peptides such as, for example, soluble peptides, including but not limited to, Ig-tailed fusion peptides, and members of random peptide libraries; (see, e.g., Lam, et al., 1991, Nature 354, 82-84; Houghten, et al., 1991, Nature 354, 84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72, 767-778), antibodies (including, but not limited to, polyclonal, monoclonal, humanized, anti-idiotypic, chimeric or single chain antibodies, and FAb, F(ab′)2, and FAb expression library fragments, and epitope-binding fragments thereof), antisense RNA, ribozymes, and small organic or inorganic molecules. Such compounds may further comprise compounds, in particular drugs or members of classes or families of drugs, known to ameliorate or exacerbate the symptoms of an immune disorder such as PAPA syndrome by acting on the cellular immune response signaling pathways. Compounds identified via assays such as those described herein may be useful, for example, in compensating for the mutation in CD2BP1 in the treatment or management of immune disorders such as PAPA syndrome.

In vitro systems may be designed to identify compounds capable of binding the mutant CD2BP1 gene products of the invention. The principle of the assays used to identify compounds that bind to the mutant CD2BP1 gene product involves preparing a reaction mixture of the CD2BP1 gene product and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring CD2BP1 gene product or the test substance onto a solid phase and detecting CD2BP1 gene product/test compound complexes anchored on the solid phase at the end of the reaction. In one embodiment of such a method, the CD2BP1 gene product may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for CD2BP1 gene product or the test compound to anchor any complexes formed in solution, and a labeled antibody specific for the other component of the possible complex to detect anchored complexes.

Any method suitable for detecting protein-protein interactions may be employed for identifying mutant CD2BP1 protein-protein interactions to identify intracellular binding partners. Among the traditional methods that may be employed are co-immunoprecipitation, cross-linking and co-purification through gradients or chromatographic columns. Utilizing procedures such as these allows for the identification of proteins, including intracellular proteins, that interact with mutant CD2BP1 gene products. Once isolated, such a protein can be identified and can be used in conjunction with standard techniques, to identify proteins with which it interacts. For example, at least a portion of the amino acid sequence of a protein that interacts with the CD2BP1 gene product can be ascertained using techniques well known to those of skill in the art, such as via the Edman degradation technique (see, e.g., Creighton, 1983, “Proteins: Structures and Molecular Principles,” W. H. Freeman & Co., N.Y., pp.34-49). The amino acid sequence obtained may be used as a guide for the generation of oligonucleotide mixtures that can be used to screen for gene sequences encoding such proteins. Screening may be accomplished, for example, by standard hybridization or PCR techniques. Techniques for the generation of oligonucleotide mixtures and the screening are well-known. (See, e.g., Ausubel, supra, and 1990, “PCR Protocols: A Guide to Methods and Applications,” Innis, et al., eds. Academic Press, Inc., New York).

Additionally, methods may be employed that result in the simultaneous identification of genes that encode for proteins that interact with wild type or mutant CD2BP1 genes or gene products. These methods include, for example, probing expression libraries with labeled CD2BP1 protein, and using CD2BP1 protein in a manner similar to the well known technique of antibody probing of lambda gtll libraries.

One method that detects protein interactions in vivo, the two-hybrid system, is described in detail for illustration only and not by way of limitation. One version of this system has been described (Chien, et al., 1991, Proc. Natl. Acad. Sci. U.S.A., 88, 9578-9582) and is commercially available from Clontech (Palo Alto, Calif.). Briefly, utilizing such a system, plasmids are constructed that encode two-hybrid proteins: one consists of the DNA-binding domain of a transcription activator protein fused to the CD2BP1 gene product and the other consists of the transcription activator protein's activation domain fused to an unknown protein that is encoded by a cDNA that has been recombined into this plasmid as part of a cDNA library. The DNA-binding domain fusion plasmid and the cDNA library are co-transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two-hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, mutation containing CD2BP1 gene products may be used as the bait gene product. Total genomic or cDNA sequences are fused to the DNA encoding an activation domain. This library and a plasmid encoding a hybrid of a bait CD2BP1 gene product fused to the DNA-binding domain are co-transformed into a yeast reporter strain, and the resulting transformants are screened for those that express the reporter gene. For example, and not by way of limitation, a bait CD2BP1 gene sequence, such as the open reading frame of the CD2BP1 gene, can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait CD2BP1 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait CD2BP1 gene-GAL4 fusion plasmid into a yeast strain that contains a lacZ gene driven by a promoter that contains GAL4 activation sequence. A cDNA encoded protein, fused to GAL4 transcriptional activation domain, that interacts with bait CD2BP1 gene product will reconstitute an active GAL4 protein and thereby drive expression of the HIS3 gene. Colonies that express HIS3 can be detected by their growth on petri dishes containing semi-solid agar based media lacking histidine. The cDNA can then be purified from these strains, and used to produce and isolate the bait CD2BP1 gene-interacting protein using techniques routinely practiced in the art.

CD2BP1 gene products of the invention may, in vivo, interact with one or more macromolecules, including intracellular macromolecules, such as proteins. Such macromolecules may include, but are not limited to, nucleic acid molecules and those proteins identified via methods such as those described above, as well as proteins known to interact with the wild-type CD2BP1 such as the cytoplasmic tail of CD2 or PTP PEST, for example. For purposes of this disclosure, the macromolecules are referred to herein as “binding partners.” Compounds that disrupt mutant CD2BP1 binding in this way may be useful in regulating the activity of the CD2BP1 gene product, especially mutant CD2BP1 gene products. Such compounds may include, but are not limited to molecules such as peptides, and the like, which would be capable of gaining access to a CD2BP1 gene product.

The basic principle of the assay systems used to identify compounds that interfere with the interaction between the CD2BP1 gene product and its binding partner or partners involves preparing a reaction mixture containing the CD2BP1 gene product, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of CD2BP1 gene product and its binding partner. Control reaction mixtures are incubated without the test compound or with a placebo. The formation of any complexes between the CD2BP1 gene protein and the binding partner is then detected. The formation of a complex in the control reaction, but not in the reaction mixture containing the test compound, indicates that the compound interferes with the interaction of the CD2BP1 gene protein and the interactive binding partner. Additionally, complex formation within reaction mixtures containing the test compound and normal CD2BP1 gene protein may also be compared to complex formation within reaction mixtures containing the test compound and a mutant CD2BP1 gene protein. This comparison may be important in those cases wherein it is desirable to identify compounds that disrupt interactions of mutant but not wild-type CD2BP1 gene proteins.

The assay for compounds that interfere with the interaction of the CD2BP1 gene products and binding partners can be conducted in a heterogeneous or homogeneous format. Heterogeneous assays involve anchoring either the CD2BP1 gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the CD2BP1 gene products and the binding partners, e.g., by competition, can be identified by conducting the reaction in the presence of the test substance; i.e., by adding the test substance to the reaction mixture prior to or simultaneously with the CD2BP1 gene protein and interactive intracellular binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the CD2BP1 gene product or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the CD2BP1 gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of the mutant CD2BP1 gene protein and the interactive binding partner is prepared in which either the CD2BP1 gene product or its binding partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances that disrupt CD2BP1 gene protein/binding partner interaction can be identified.

In a particular embodiment, the mutant CD2BP1 gene product can be prepared for immobilization using recombinant DNA techniques. For example, the CD2BP1 coding region can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with the radioactive isotope ¹²⁵I, for example, by methods routinely practiced in the art. In a heterogeneous assay, e.g., the GST-CD2BP1 fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the CD2BP1 gene protein and the interactive binding partner can be detected by measuring the amount of radioactivity that remains associated with the glutathione-agarose beads. A successful inhibition of the interaction by the test compound will result in a decrease in measured radioactivity.

Alternatively, the GST-CD2BP1 gene fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the CD2BP1 gene product/binding partner interaction can be detected by adding the labeled antibody and measuring the radioactivity associated with the beads.

In another embodiment of the invention, these same techniques can be employed using peptide fragments that correspond to the binding domains of the CD2BP1 protein and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the segments can be engineered to express peptide fragments of the protein, which can then be tested for binding activity and purified or synthesized. For example, and not by way of limitation, a mutant CD2BP1 gene product can be anchored to a solid material as described by making a GST-CD2BP1 fusion protein and allowing it to bind to glutathione agarose beads. The interactive binding partner obtained can be labeled with a radioactive isotope, such as ³⁵S, and cleaved with a proteolytic enzyme such as trypsin. Cleavage products can then be added to the anchored GST-CD2BP1 fusion protein and allowed to bind. After washing away unbound peptides, labeled bound material, representing the binding partner binding domain, can be eluted, purified, and analyzed for amino acid sequence by well-known methods. Peptides so identified can be produced synthetically or fused to appropriate facilitative proteins using recombinant DNA technology.

In a further aspect of the invention, animal-based systems or models for a CD2BP1 mediated immune disorder, which may include, for example, mutant CD2BP1 or mutant PSTPIP expressing mice, may be used to identify compounds capable of ameliorating symptoms of the disorder. Such animal models may be used as test substrates for the identification of drugs, pharmaceuticals, therapies and interventions that may be effective in treating such disorders. For example, animal models may be exposed to a compound suspected of exhibiting an ability to ameliorate symptoms, at a sufficient concentration and for a sufficient time to elicit such an amelioration of symptoms of an immune disorder in the exposed animals. The response of the animals to the exposure may be monitored by assessing the reversal of such symptoms. With regard to intervention, any treatments that reverse any aspect of symptoms of an immune disorder should be considered as candidates for human therapeutic intervention in such a disorder. Dosages of test agents may be determined by deriving dose-response curves, which are well known to those of skill in the art.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

The following example describes the identification of the chromosomal region that harbors the causative gene for familial recurrent arthritis (FRA), as well as PAPA syndrome, via a genome wide linkage scan in an extended kindred with FRA. A genome wide linkage survey of this extended kindred localizes the causative gene to human chromosome 15q22-24.

A three generation family was ascertained in which nine members were diagnosed with juvenile idiopathic arthritis. In this family the disease was of very early onset and included episodic inflammation leading to eventual destruction of joints, muscle, and skin. The simple inheritance pattern and number of affected individuals in this family permitted the performance of a genome-wide linkage scan to localize the causative allele. This allele was contemplated to likely play a fundamental role in the immune cascade leading to tissue destruction in this disorder.

The proband, FRA1-1, presented as a 5-year-old boy with a history of recurrent joint swelling and cystic skin lesions since infancy. Arthritis was characteristically intermittent and migratory and led to the accumulation of sterile pyogenic material within the joint space if left untreated. It followed a mono-articular pattern (rarely more than one joint affected during flares). It involved primarily the elbows, knees, and ankles, although small joints were occasionally affected. There was no history of recurrent infections or granuloma formation. Family history was remarkable for the presence of similar symptoms in the patient's father and twin brothers, paternal aunt and two cousins. The father reported a marked improvement in joint symptoms after puberty, with the subsequent appearance of severe acne.

The proband's initial physical examination revealed a slightly pale boy in the 25th percentile for weight and 5th percentile for height. In general the examination was unremarkable except for the presence of 2×2 cm scar on the antero-lateral aspect of the right shoulder and a 50% loss of function of the left elbow, which displayed a 2×3 cm scar from a previous synovectomy. At the time of this initial visit, he was on low dose daily prednisone (5 mg) and nonsteroidal anti-inflammatory drugs (NSAIDs). During the next four years, this patient continued presenting intermittent episodes of arthritis initially characterized by tenderness, erythema, and non-fluctuating joint swelling. These episodes either responded to the administration of intra-articular steroids, or evolved in a matter of days into a fluctuating stage with the accumulation of pseudo-purulent, sterile material that required surgical draining for healing to occur. During these episodes the patient remained afebrile, and laboratory tests characteristically showed anemia, normal white blood cell (WBC) count, and differential, elevated aldolase and erythrocyte sedimentation rate (ESR). Laboratory values typically returned to normal levels in between the arthritis episodes, except for aldolase, which remained elevated.

Cultures of the synovial fluid and tissue for bacteria, mycobacteria and fungi were repeatedly negative. Anti-nuclear antibodies (ANA), rheumatoid factor (RF), CH₅₀, C3, C4, and immunoglobulin levels were normal. Peripheral blood T, B, NK and monocyte cell counts as well as T and B cell subpopulations were normal according to flow cytometry markers (CD3, CD4, CD8, CD14, CD16, CD19, CD20, CD56). Nitroblue tetrazolium (NBT) and bactericidal capacity were normal. Synovial tissue biopsy revealed polymorphonuclear infiltrate, and immunofluorescence analysis failed to disclose the presence of immunoglobulin or complement deposits. At the age of eight, the patient began presenting erythematous/violaceous papular cutaneous lesions on the legs and arms that would evolve into sterile pustules and eventually into ulcers. This patient continued presenting recurrent attacks of joint and skin inflammation in spite of treatment with daily PO prednisone and intermittent intra-articular and high dose intravenous steroids. Trials of subcutaneous methotrexate, and PO hydroxychloroquine were unsuccessful. Surgical debridement of involved joints, especially the elbows, was required on several occasions in order to control the inflammatory process. At the time of this disclosure, this patient had normal joint function with the exception of a 25% and a 50% reduction in right and left elbow mobility respectively.

Two twin monozygotic male siblings of the proband had a similar although milder clinical history. They also had recurrent joint swelling since infancy. As in the proband, swelling primarily involved single joints including the elbows, knees and ankles; however, no skin lesions compatible with pyoderma gangrenosum were noted. Laboratory results showed leukocytosis and elevated ESR during flares. Anemia was mild, and aldolase remained persistently elevated. These patients were treated with oral prednisone, and periodic local steroid injections to control their symptoms.

DNA was isolated from 10 mL whole blood by a differential lysis procedure (Boehringer Mannheim catalog no. 1667327). Fluorescently-labeled microsatellite markers were amplified with the polymerase chain reaction (PCR) in a Perkin-Elmer 9600 or M3 Research Tetrad thermocycler. Reactions were performed in 15 uL containing 1.5 mM MgCl₂, 500 mM KCl, 0.25 mM each dNTP, 1.25 pmol each primer, 0.5 U Taq polymerase (Perkin Elmer Cetus), and 100 ng patient DNA. Amplification conditions were 95° C. 5 minutes, then 28 cycles, 94° C. 30s, 56° C. 30s, 72° C. 30s, with a final extension of 6 minutes, 72° C. PCR products from each individual were pooled, mixed with loading dye and TAMRA 500 size standards (Perkin Elmer Cetus) and denatured for 5 minutes, 95° C. Alleles were separated by electrophoresis in a 12 or 36 cm 4.65% denaturing acrylamide gel at 760V for 2-3 hours with an ABI PRISM 377 DNA Sequencer/Genotyper (Applied Biosystems). Results were analyzed by the GeneScan (version 2.01) and GENOTYPER software packages (Applied Biosystems).

All alleles called by GENOTYPER were examined independently by two individuals. Uninformative markers were replaced with informative ones wherever possible. Two point lod scores were calculated by FASTLINK and MLINK versions of the LINKAGE program (Lathrop et al., 1984, Proc Natl Acad Sci U.S.A. 81:3443). Given that FRA is clearly a rare disorder, but of unknown prevalence, linkage calculations were performed incorporating gene frequencies of both 0.0001 and 0.00001. When available allele frequencies were obtained from the Marshfield Database (http://www.marshmed.org/genetic/); for all other loci equal allele frequencies were used.

Whole blood lymphocytes from the proband's affected father were found to be karyotypically normal. Localization of the susceptibility gene therefore required a genome-wide linkage scan. A two-stage genome wide scan was initiated in which 169 polymorphic autosomal loci at an average resolution of approximately 25 cM were first genotyped in twelve family members including eight affected individuals. Pairwise lod scores using a model of dominant inheritance with 100% penetrance provided strongest evidence for linkage at D15S655 and D15SI75. In the second stage of the survey an additional 102 polymorphic autosomal loci were genotyped in the original family as well as a newly-ascertained affected cousin, FRA-16, to provide finer mapping of all regions of potential linkage. Significant evidence of linkage was obtained only at D15S175 (Zₘₐₓ=3.23 at 0ₘₐₓ=0.0010), and increasing the estimated gene frequency from 0.00001 to 0.0001 did not significantly alter this result. Refinement of this region with additional loci generated the two point lod scores. Although lod scores for all loci were also generated with penetrances of 80 and 90%, the highest lod scores were obtained using 100% penetrance. The maximum lod score was obtained with D15S211 (Zₘₐₓ=3.27, 0ₘₐₓ=0.0010). Critical recombinants in this family localized FRA to a region approximately 20 cM flanked proximally by D15S983 and distally by D15S127 on chromosome 15.

### EXAMPLE 2

The following example describes the isolation and identification of two mutant alleles in the gene encoding the CD2 binding protein, CD2BP1. The nucleic acid sequence of CD2BP1 is described herein as SEQ ID NO:18, long form is available as Genbank accession #AF038603. SEQ ID NO:18 is as follows:

SEQ ID NO:18

| | |
|---|---|
| 1 | atgatgcccc agctgcagtt caaagatgcc ttttggtgca |

gggacttcac agcccacacg

| | |
|---|---|
| 61 | ggctacgagg tgctgctgca gcggcttctg gatggcagga |

agatgtgcaa agacatggag

| | |
|---|---|
| 121 | gagctactga ggcagagggc ccaggcggag gagcggtacg |

ggaaggagct ggtgcagatc

| | |
|---|---|
| 181 | gcacggaagg caggtggcca gacggagatc aactccctga |

gggcctcctt tgactccttg

| | |
|---|---|
| 241 | aagcagcaaa tggagaatgt gggcagctca cacatccagc |

tggccctgac cctgcgtgag

| | |
|---|---|
| 301 | gagctgcgga gtctcgagga gtttcgtgag aggcagaagg |

agcagaggaa gaagtatgag

| | |
|---|---|
| 361 | gccgtcatgg accgggtcca gaagagcaag ctgtcgctct |

acaagaaggc catggagtcc

| | |
|---|---|
| 421 | aagaagacat acgagcagaa gtgccgggac gcggacgacg |

cggagcaggc cttcgagcgc

| | |
|---|---|
| 481 | attagcgcca acggccacca gaagcaggtg gagaagagtc |

agaacaaagc caggcagtgc

| | |
|---|---|
| 541 | aaggactcgg ccaccgaggc agagcgggta tacaggcaga |

gcattgcgca gctggagaag

| | |
|---|---|
| 601 | gtccgggctg agtgggagca ggagcaccgg accacctgtg |

aggcctttca gctgcaagag

| | |
|---|---|
| 661 | tttgaccggc tgaccattct ccgcaacgcc ctgtgggtgc |

acagcaacca gctctccatg

| | |
|---|---|
| 721 | cagtgtgtca aggatgatga gctctacgag gaagtgcggc |

tgacgctgga aggctgcagc

| | |
|---|---|
| 781 | atagacgccg acatcgacag tttcatccag gccaagagca |

cgggcacaga gccccccgct

| | |
|---|---|
| 841 | ccggtgccct accagaacta ttacgatcgg gaggtcaccc |

cgctgaccag cagccctggc

| | |
|---|---|
| 901 | atacagccgt cctgcggcat gataaagagg ttctctggac |

tgctgcacgg aagtcccaag

| | |
|---|---|
| 961 | accacttcgt tggcagcttc tgctgcgtcc acagagaccc |

tgacccccac ccccgagcgg

| | |
|---|---|
| 1021 | aatgagggtg tctacacagc catcgcagtg caggagatac |

agggaaaccc ggcctcacca

| | |
|---|---|
| 1081 | gcccaggagt accgggcgct ctacgattat acagcgcaga |

acccagatga gctggacctg

| | |
|---|---|
| 1141 | tccgcgggag acatcctgga ggtgatcctg gaaggggagg |

atggctggtg gactgtggag

| | |
|---|---|
| 1201 | aggaacgggc agcgtggctt cgtccctggt tcctacctgg |

agaagctttg aggaagggcc

| | |
|---|---|
| 1261 | aggagcccct tcggacctgc cctgccagtg gagccagcag |

tgcccccagc actgtcccca

| | |
|---|---|
| 1321 | ccttgctagg gcccagaacc aagcgtcccc cagccccgag |

agggagcctg tcgtctccca

| | |
|---|---|
| 1381 | gggaataaag gagtgcgttc tgttctaaaa aaaaaaaaaa |

aaaaaaaa

The mutation at nucleic acid 748 in the sequence described herein as SEQ ID NO:19 is shown herein to be associated with familial recurrent arthritis (E250Q). The amino acid sequence of the E250Q mutant is designated herein as SEQ ID NO:20. The mutation at nucleic acid 688 in the sequence described herein as SEQ ID NO:21 is shown herein to be associated with PAPA syndrome (A230T). The amino acid sequence of the A230T mutant is designated herein as SEQ ID NO:22. FRA and PAPA syndrome thus are the same disease, and are referred to collectively as PAPA syndrome.

A partial physical and transcription map of the FRA critical region was constructed in silico by assembling BAC clones and known genes from public databases. BAC sequences found in Genbank were analyzed for coding regions by the NIX gene prediction software suite (Human Genome Mapping Protocol (HGMP) Website), generating precise linear placement of several genes. Other genes were integrated from the G3 and GB4 radiation hybrid maps (NCBI Website). Arthritis and ulcerative skin lesions found in individuals affected with FRA appear to have an autoimmune aetiology; therefore priority was given to positional candidate genes expressed in hematopoietic tissues and/or those that were known to function in the inflammatory response. These genes were analyzed for mutations in affected individuals from family FRA1 by either direct DNA sequencing or denaturing high performance liquid chromatography (DHPLC) to detect heteroduplex formation. Several genes were excluded by these analyses.

The CD2 binding protein 1 (CD2BP1) gene was considered a candidate for mutation screening for several reasons. It was originally identified by a yeast two-hybrid interaction trap system designed to detect proteins interacting with the cytoplasmic tail of the CD2 protein. CD2 is found on the surface of virtually all T cells and natural killer (NK) cells, where it mediates adhesion to target cells and antigen presenting cells (APCs). CD2BP1 expression is restricted almost exclusively to hematopoietic tissues as detected by Northern analysis, and evidence suggests that the CD2BP1 protein may function as a negative regulator of CD2 adhesion. In addition, the mouse homolog of CD2BP1, PSTPIP, has been demonstrated by the yeast two-hybrid system to interact with the protein which is deficient in Wiskott-Aldrich syndrome, an X-linked disorder marked by a compromised immune deficiency.

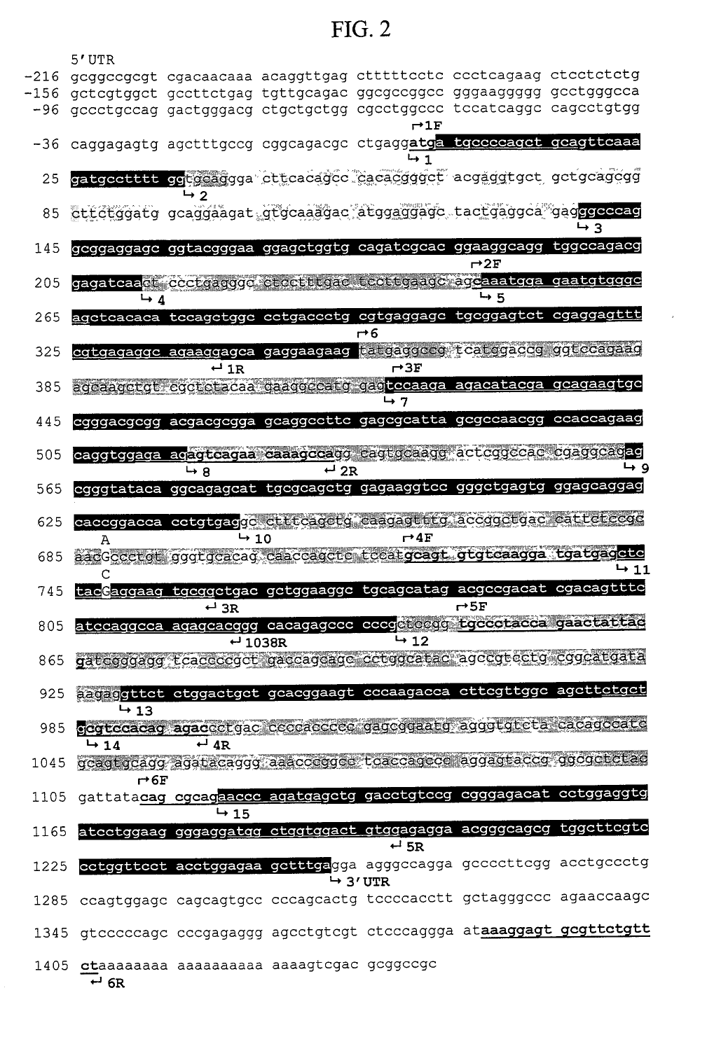

PCR primers were designed from the CD2BP1 cDNA sequence to amplify the cDNA in six overlapping fragments (see FIG. 2). These amplimers were generated by RT-PCR of total RNA from affected family members FRA1-1 and FRA1-10 EBV lymphoblastoid cell lines. Analysis by DHPLC detected heteroduplex formation in the fragment amplified by primers 4F and 4R (SEQ ID NO:10 and SEQ ID NO:11, respectively), suggesting the presence of a sequence variant in one allele. Interestingly, alternative splicing of this region in activated T cells has been reported to generate two transcripts differing by 57 nucleotides (Li et al., EMBO 17(24):7320-7336, 1998). However, only the longer version was detected in amplified patient or control lymphoblastoid cDNA. The same analysis of a portion of this fragment amplified from patient cDNA again detected a potential sequence variant. The exon/intron boundaries of CD2BP1 were derived by alignment of the cDNA sequence with human genomic sequence from the Celera database (see FIG. 2). This analysis indicated that the CD2BP1 gene contains 15 exons, with the alternatively spliced region encompassing exon 12. Primers were designed to amplify each of the 15 identified exons from genomic DNA. (FIG. 2).

An intronic primer upstream of exon 11 was designed to amplify the variant-containing region from genomic DNA of FRA family members. DHPLC analysis of this 189 base pair amplimer, designated as 5′ exon 11, demonstrated that the variant co-segregates with disease in the FRA family. Sequence analysis identified a G-to-C transversion at nucleotide 748 of the CD2BP1 cDNA. Sequencing of the cloned 5′ exon 11 amplimer from affected individual FRA1-1 revealed 34% of clones with the G-to-C mutation and 66% wild type. The G-to-C transversion at nucleotide 748 of the CD2BP1 cDNA (SEQ ID NO:19) creates a E250Q variant that co-segregates with disease in the FRA family. DHPLC analysis of the 5′ exon 11 amplimer of 228 control chromosomes from a panel of unrelated Caucasian Americans revealed no heterozygous sequence variants; 72 of these wild-type chromosomes were confirmed by DNA sequencing. In addition, no heteroduplexes were detected by DHPLC analysis of controls mixed with wild type 5′ exon 11 amplimer, suggesting the absence of any homozygous changes from the wild-type sequence in this region in control individuals.

The E250Q mutant CD2BP1 nucleic acid sequence follows with the mutation at nucleotide 748, which is capitalized and shown in bold:

SEQ ID NO:19

| | |
|---|---|
| 1 | atgatgcccc agctgcagtt caaagatgcc ttttggtgca |

gggacttcac agcccacacg

| | |
|---|---|
| 61 | ggctacgagg tgctgctgca gcggcttctg gatggcagga |

agatgtgcaa agacatggag

| | |
|---|---|
| 121 | gagctactga ggcagagggc ccaggcggag gagcggtacg |

ggaaggagct ggtgcagatc

| | |
|---|---|
| 181 | gcacggaagg caggtggcca gacggagatc aactccctga |

gggcctcctt tgactccttg

| | |
|---|---|
| 241 | aagcagcaaa tggagaatgt gggcagctca cacatccagc |

tggccctgac cctgcgtgag

| | |
|---|---|
| 301 | gagctgcgga gtctcgagga gtttcgtgag aggcagaagg |

agcagaggaa gaagtatgag

| | |
|---|---|
| 361 | gccgtcatgg accgggtcca gaagagcaag ctgtcgctct |

acaagaaggc catggagtcc

| | |
|---|---|
| 421 | aagaagacat acgagcagaa gtgccgggac gcggacgacg |

cggagcaggc cttcgagcgc

| | |
|---|---|
| 481 | attagcgcca acggccacca gaagcaggtg gagaagagtc |

agaacaaagc caggcagtgc

| | |
|---|---|
| 541 | aaggactcgg ccaccgaggc agagcgggta tacaggcaga |

gcattgcgca gctggagaag

| | |
|---|---|
| 601 | gtccgggctg agtgggagca ggagcaccgg accacctgtg |

aggcctttca gctgcaagag

| | |
|---|---|
| 661 | tttgaccggc tgaccattct ccgcaacgcc ctgtgggtgc |

acagcaacca gctctccatg

| | |
|---|---|
| 721 | cagtgtgtca aggatgatga gctctacCag gaagtgcggc |

tgacgctgga aggctgcagc

| | |
|---|---|
| 781 | atagacgccg acatcgacag tttcatccag gccaagagca |

cgggcacaga gccccccgct

| | |
|---|---|
| 841 | ccggtgccct accagaacta ttacgatcgg gaggtcaccc |

cgctgaccag cagccctggc

| | |
|---|---|
| 901 | atacagccgt cctgcggcat gataaagagg ttctctggac |

tgctgcacgg aagtcccaag

| | |
|---|---|
| 961 | accacttcgt tggcagcttc tgctgcgtcc acagagaccc |

tgacccccac ccccgagcgg

| | |
|---|---|
| 1021 | aatgagggtg tctacacagc catcgcagtg caggagatac |

agggaaaccc ggcctcacca

| | |
|---|---|
| 1081 | gcccaggagt accgggcgct ctacgattat acagcgcaga |

acccagatga gctggacctg

| | |
|---|---|
| 1141 | tccgcgggag acatcctgga ggtgatcctg gaaggggagg |

atggctggtg gactgtggag

| | |
|---|---|
| 1201 | aggaacgggc agcgtggctt cgtccctggt tcctacctgg |

agaagctttg aggaagggcc

| | |
|---|---|
| 1261 | aggagcccct tcggacctgc cctgccagtg gagccagcag |

tgcccccagc actgtcccca

| | |
|---|---|
| 1321 | ccttgctagg gcccagaacc aagcgtcccc cagccccgag |

agggagcctg tcgtctccca

| | |
|---|---|
| 1381 | gggaataaag gagtgcgttc tgttctaaaa aaaaaaaaaa |

aaaaaaaa

The E250Q mutant CD2BP1 amino acid sequence follows with the mutation at amino acid 250 shown in bold:

SEQ ID NO:20

MMPQLQFKDA FWCRDFTAHT GYEVLLQRLL DGRKMCKDME
ELLRQRAQAE ERYGKELVQI ARKAGGQTEI NSLRASFDSL
KQQMENVGSS HIQLALTLRE ELRSLEEFRE RQKEQRKKYE
AVMDRVQKSK LSLYKKAMES KKTYEQKCRD ADDAEQAFER
ISANGHQKQV EKSQNKARQC KDSATEAERV YRQSIAQLEK
VRAEWEQEHR TTCEAFQLQE FDRLTILRNA LWVHSNQLSM
QCVKDDELYQEVRLTLEGCS IDADIDSFIQ AKSTGTEPPA
PVPYQNYYDR EVTPLTSSPG IQPSCGMIKR FSGLLHGSPK
TTSLAASAAS TETLTPTPER NEGVYTAIAV QEIQGNPASP
AQEYRALYDY TAQNPDELDL SAGDILEVIL EGEDGWWTVE
RNGQRGFVPG SYLEKL

Similar analyses as those described above in members of a family with PAPA syndrome detected a G-to-A transition at nucleotide 688 of CD2BP1, predicted to create an A230T variant in affected individuals. This mutation was discovered to co-segregate with disease in the PAPA family discussed in the Lindor et al. reference (Lindor et al., 1997, Mayo Clin Proc 72:611). DHPLC analysis of 228 unrelated control chromosomes revealed no heterozygous sequence variants in PCR-amplified CD2BP1 exon 10; DNA sequencing revealed no differences from the published sequence in 72 of these controls. As anticipated from the original DHPLC results, no changes from wild-type were detected in the remaining exons amplified from genomic DNA of individuals analyzed with PAPA syndrome.

The A230T mutant CD2BP1 nucleic acid sequence follows with the mutation at nucleotide 688, which is capitalized and shown in bold:

SEQ ID NO:21

| | |
|---|---|
| 1 | atgatgcccc agctgcagtt caaagatgcc ttttggtgca |

gggacttcac agcccacacg

| | |
|---|---|
| 61 | ggctacgagg tgctgctgca gcggcttctg gatggcagga |

agatgtgcaa agacatggag

| | |
|---|---|
| 121 | gagctactga ggcagagggc ccaggcggag gagcggtacg |

ggaaggagct ggtgcagatc

| | |
|---|---|
| 181 | gcacggaagg caggtggcca gacggagatc aactccctga |

gggcctcctt tgactccttg

| | |
|---|---|
| 241 | aagcagcaaa tggagaatgt gggcagctca cacatccagc |

tggccctgac cctgcgtgag

| | |
|---|---|
| 301 | gagctgcgga gtctcgagga gtttcgtgag aggcagaagg |

agcagaggaa gaagtatgag

| | |
|---|---|
| 361 | gccgtcatgg accgggtcca gaagagcaag ctgtcgctct |

acaagaaggc catggagtcc

| | |
|---|---|
| 421 | aagaagacat acgagcagaa gtgccgggac gcggacgacg |

cggagcaggc cttcgagcgc

| | |
|---|---|
| 481 | attagcgcca acggccacca gaagcaggtg gagaagagtc |

agaacaaagc caggcagtgc

| | |
|---|---|
| 541 | aaggactcgg ccaccgaggc agagcgggta tacaggcaga |

gcattgcgca gctggagaag

| | |
|---|---|
| 601 | gtccgggctg agtgggagca ggagcaccgg accacctgtg |

aggcctttca gctgcaagag

| | |
|---|---|
| 661 | tttgaccggc tgaccattct ccgcaacAcc ctgtgggtgc |

acagcaacca gctctccatg

| | |
|---|---|
| 721 | cagtgtgtca aggatgatga gctctacgag gaagtgcggc |

tgacgctgga aggctgcagc

| | |
|---|---|
| 781 | atagacgccg acatcgacag tttcatccag gccaagagca |

cgggcacaga gccccccgct

| | |
|---|---|
| 841 | ccggtgccct accagaacta ttacgatcgg gaggtcaccc |

cgctgaccag cagccctggc

| | |
|---|---|
| 901 | atacagccgt cctgcggcat gataaagagg ttctctggac |

tgctgcacgg aagtcccaag

| | |
|---|---|
| 961 | accacttcgt tggcagcttc tgctgcgtcc acagagaccc |

tgacccccac ccccgagcgg

| | |
|---|---|
| 1021 | aatgagggtg tctacacagc catcgcagtg caggagatac |

agggaaaccc ggcctcacca

| | |
|---|---|
| 1081 | gcccaggagt accgggcgct ctacgattat acagcgcaga |

acccagatga gctggacctg

| | |
|---|---|
| 1141 | tccgcgggag acatcctgga ggtgatcctg gaaggggagg |

atggctggtg gactgtggag

| | |
|---|---|
| 1201 | aggaacgggc agcgtggctt cgtccctggt tcctacctgg |

agaagctttg aggaagggcc

| | |
|---|---|
| 1261 | aggagcccct tcggacctgc cctgccagtg gagccagcag |

tgcccccagc actgtcccca

| | |
|---|---|
| 1321 | ccttgctagg gcccagaacc aagcgtcccc cagccccgag |

agggagcctg tcgtctccca

| | |
|---|---|
| 1381 | gggaataaag gagtgcgttc tgttctaaaa aaaaaaaaaa |

aaaaaaaa

The A230T mutant CD2BP1 amino acid sequence follows with the mutation at amino acid 230 shown in bold:

SEQ ID NO:22

MMPQLQFKDA FWCRDFTAHT GYEVLLQRLL DGRKMCKDME
ELLRQRAQAE ERYGKELVQI ARKAGGQTEI NSLRASFDSL
KQQMENVGSS HIQLALTLRE ELRSLEEFRE RQKEQRKKYE
AVMDRVQKSK LSLYKKAMES KKTYEQKCRD ADDAEQAFER
ISANGHQKQV EKSQNKARQC KDSATEAERV YRQSIAQLEK
VRAEWEQEHR TTCEAFQLQE FDRLTILRNT LWVHSNQLSM
QCVKDDELYE EVRLTLEGCS IDADIDSFIQ AKSTGTEPPA
PVPYQNYYDR EVTPLTSSPG IQPSCGMIKR FSGLLHGSPK
TTSLAASAAS TETLTPTPER NEGVYTAIAV QEIQGNPASP
AQEYRALYDY TAQNPDELDL SAGDILEVIL EGEDGWWTVE
RNGQRGFVPG SYLEKL

Amino acids 123-288 of the CD2BP1 protein, containing both E250Q and A230T, bear approximately 30% similarity to the Schizosaccharomyces pombe CDC15 protein, a phosphoprotein involved in organization of the actin ring and cleavage furrow formation during cytokinesis. The murine homolog PSTPIP has been demonstrated to co-localize with the cortical actin cytoskeleton, lamellipodia, and the cytokinetic cleavage furrow, and 3T3 cells in which the protein has been overexpressed form extended filopodia. In addition, overexpression of the PSTPIP protein in S. pombe inhibits cytokinesis. These observations led to the proposal that the murine homolog PSTPIP is functionally homologous to CDC15. Like CDC15, CD2BP1 contains a helical domain and a carboxy terminal SH3 region; in addition, in between these two regions is a sequence rich in proline, glutamic acid, serine, and threonine (PEST) residues. The PEST sequence found in the alternatively spliced sequence between these domains is of variable length. The SH3 domain of CD2BP1 or PSTPIP1 was previously shown to interact with CD2, c-Abl, and WASP, whereas the PTP PEST interaction domain was mapped within the region of CD2BP1 homologous to CDC15, and potentially encompasses the E250Q and A230T variants detected.

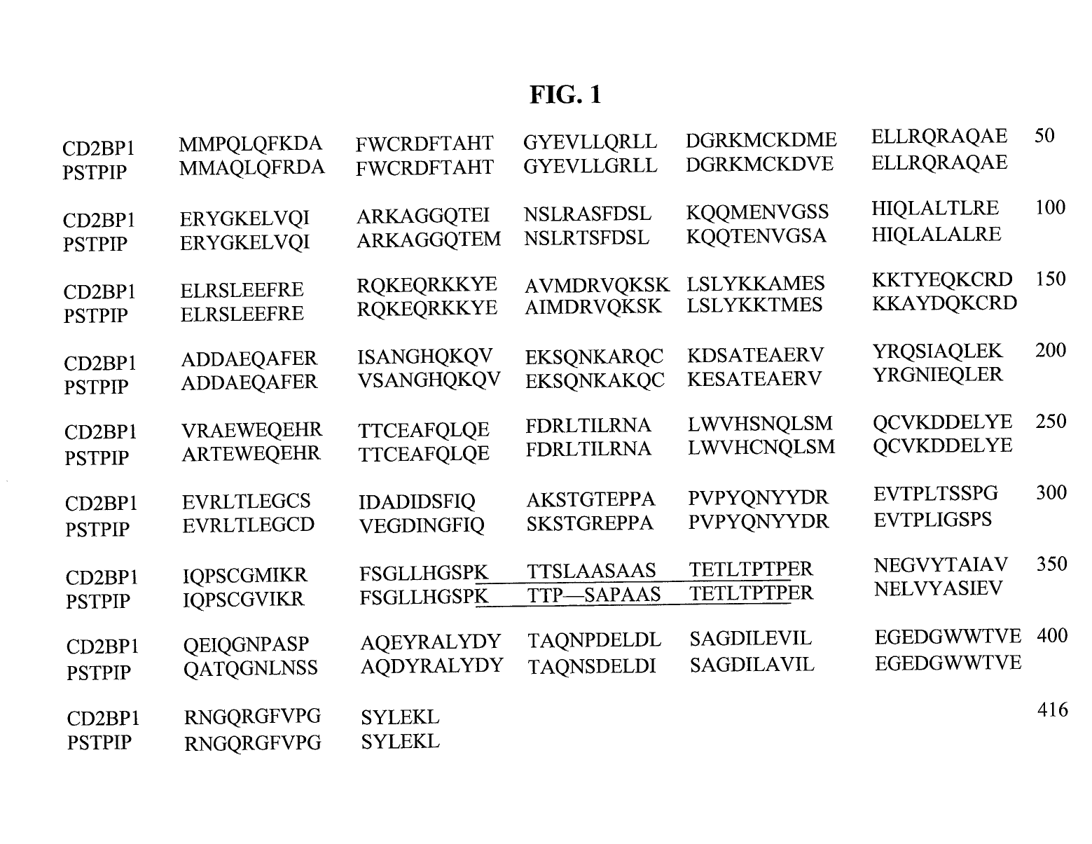

The G748C missense mutation in CD2BP1 is predicted to change negatively charged glutamic acid (E) 250 to a polar uncharged glutamine (Q); this residue is conserved in both the murine protein PSTPIP, as well as within the S. pombe CDC15 gene (FIG. 1). The G748C annotation denotes a G to C transversion at position 748 of the native sequence (Genbank Accession #AF038603, SEQ ID NO:18), and the mutant sequence which is designated herein as SEQ ID NO:19. Interestingly, the G688A missense mutation in CD2BP1 is predicted to change nonpolar alanine (A) 230 to another polar uncharged amino acid, threonine (T); this residue is also conserved in the murine protein PSTPIP (FIG. 1). The conserved alanine at position 230 in the mouse PSTPIP protein sequence is 2 amino acids away from the tryptophan previously identified as critical for PTP HSCF binding in the mouse (Dowbenko et al., J. Biol Chem. 273(2):989-996,1998). This mutation is also within the CDC15 domain of CD2BP1, which also contains the conserved tryptophan. The G688A annotation denotes a G to A transition at position 688 of the native sequence (Genbank Accession #AF038603, SEQ ID NO:18), and the mutant sequence which is designated herein as SEQ ID NO:21.

Methods

PCR amplification. Whole cell RNA was isolated from Epstein Barr virus-transformed lymphoblastoid cell lines established from patients FRA1-1 and FRA1-10 by a modified guanidium thiocyanate lysis procedure (Roche Boehringer Mannheim cat. number 1667 165). RNA was converted to cDNA by both random priming and dT priming with reverse transcriptase (Roche Boehringer Mannheim cat. number 1483 188). CD2BP1 was produced in six fragments by amplification of cDNA with the following primers:

SEQ ID NO:4

| | |
|---|---|
| CD2BP1-1F: | ATG ATG CCC CAG CTG CA,; |

SEQ ID NO:5

| | |
|---|---|
| CD2BP1-1R: | CCT TCT GCC TCT CAC GAA AC,; |

SEQ ID NO:6

| | |
|---|---|
| CD2BP1-2F: | CAA ATG GAG AAT GTG GGC AG,; |

SEQ ID NO:7

| | |
|---|---|
| CD2BP1-2R: | TGG CTT TGT TCT GAC TCT TCT,; |

SEQ ID NO:8

| | |
|---|---|
| CD2BP1-3F: | CCA AGA AGA CAT ACG AGC AGA AG,; |

SEQ ID NO:9

| | |
|---|---|
| CD2BP1-3R: | CCG CAC TTC CTC GTA GAG,; |

SEQ ID NO:10

| | |
|---|---|
| CD2BP1-4F: | GCA GTG TGT CAA GGA TGA TG,; |

SEQ ID NO:11

| | |
|---|---|
| CD2BP1-4R: | GTC TCT GTG GAC GCA GCA G,; |

SEQ ID NO:12

| | |
|---|---|
| CD2BP1-5F: | TGC CCT ACC AGA ACT ATT ACG,; |

SEQ ID NO:13

| | |
|---|---|
| CD2BP1-5R: | CCA CAG TCC ACC AGC CAT,; |

SEQ ID NO:14

| | |
|---|---|
| CD2BP1-6F: | CAG CGC AGA ACC CAG ATG A,; |

SEQ ID NO:15

| | |
|---|---|
| CD2BP1-6R: | AGA ACA GAA CGC ACT CCT TT,. |

The 5′ exon 11 fragment was amplified from genomic DNA with CD2BP1 exon 11-109F: CAC AAT GGC CTG TGA GGA G, SEQ ID NO:16, and CD2BP1-1038R: CGT GCT CTT GGC CTG GAT, SEQ ID NO:17. All amplimers were generated in the polymerase chain reaction (PCR) in a Perkin-Elmer 9600 or MJ Research Tetrad thermocycler. Reactions were performed in 25 μl containing 1.5 mM MgCl₂, 500 mM KCl, 0.25 mM each dNTP, 1.0 pmol each primer, 0.5 U Taq polymerase (Perkin Elmer Cetus), and 100 ng patient DNA. Amplification conditions were 35 cycles, 94° C. 30s, 56° C. 30s, 72° C. 3s for all fragments except 5′ exon 11 for which an annealing temperature of 55° C. was used.

Mutation analysis. Denaturing high performance liquid chromatography was performed with a WAVE machine (Transgenomics) utilizing conditions recommended by WaveMaker v3.3.3 software. CD2BP1 amplified fragments 1, 2, 3, and 5 were analyzed at an oven temperature of 65° C., fragments 4 and 5′ exon 11 at 64° C., and fragment 6 at 66° C. For sequencing, PCR products were purified from agarose gels with a purification kit (Qiagen). Amplimers were sequenced with the BigDye Terminator cycle sequencing ready reaction kit (Applied Biosystems). Sequencing products were separated by electrophoresis through a 36 cm 4.25% denaturing acrylamide gel at 760V for 3-7 hours with an ABI PRISM 377 DNA Sequencer/Genotyper (Applied Biosystems).

### EXAMPLE 3

The following example describes using yeast two-hybrid assays to determine the level of binding between PTP PEST and the mutant CD2BP1 proteins. These assays demonstrated severely reduced binding between PTP PEST and both the E250Q and A230T mutant CD2BP1 proteins. The series of yeast two-hybrid experiments used as bait a full length wild-type CD2BP1 construct cloned in-frame with the GAL4 binding domain (GAL4BD). A yeast strain expressing this fusion protein was transformed with human thymus cDNAs fused downstream of the GAL4 transcriptional activation domain (GAL4AD). In one experiment, a screen of 2×10⁵ transformants yielded twenty isolates that co-activated both the HIS3 and lacZ reporter genes were identified. These isolates were cured of the wild-type CD2BP1 GAL4BD, and re-transformed with a full length E250Q mutant CD2BP1 construct cloned in the GAL4BD plasmid. In a separate series of experiments the wild-type CD2BP1-GAL4DB was re-transformed into each cured strain to confirm that the positive interaction could be re-established. Of these, only one (clone 81 plus E250Q mutant CD2BP1-GAL4BD) failed to activate HIS3. DNA sequencing revealed that the GAL4AD plasmid from clone 81 contained the last thirty-two C-terminal amino acids (plus 3′ untranslated region) of the protein tyrosine phosphatase FTP-PEST. The failure of clone 81 to grow in the absence of histidine when in combination with the mutant construct, versus its strong growth with the wild-type CD2BP1, suggested that the E250Q change disrupts CD2BP1:PTP PEST binding. This interaction, however, was not completely abrogated.

Plating clone 81:CD2BP1 E250Q-GAL4BD cotransformants on media that does not select for interactions (−leu, −trp, +his) resulted in colonies with weak but reproducible lacZ activity, indicating low level interaction with the mutant. The interactions between clone 81 and either wild-type or E250Q mutant CD2BP1 were quantitatively assayed by measuring β-galactosidase (β-GAL) activity. Triplicate measurements resulted in an average of 100 β-GAL units for wild-type CD2BP1:clone 81 interaction compared to an average of 1.2 β-GAL units for the E250Q mutant CD2BP1:clone 81 interaction. Therefore, it appears that a residual interaction of approximately 1.2% of wild-type levels still occurs between PTP-PEST and the E250Q mutant but is insufficient to result in histidine prototrophy in the two-hybrid screen.

When the A230T mutant was transformed with clone 81 the level of β-GAL activity was reduced to less than 10% of wild-type levels, confirming that this residue is also important for PTP PEST interaction with CD2BP1. In parallel quantitative two-hybrid experiments, no detectable differences in the interactions between CD2 and wild-type, E250Q, or A230T CD2BP1 mutants was found; however, deletion of the CD2BP1 SH3 domain completely abolished this interaction, confirming the previous observation that this domain is essential for CD2 binding.

Based on the yeast two-hybrid assays, the PAPA syndrome may be caused by mutations in the CD2BP1 gene that disrupt a biologically critical binding interaction with PTP PEST. The loss of interaction between the CD2BP1 E250Q and A230T mutants and the terminal 30 amino acids of PTP PEST is consistent with the previous observation that PSTPIP1 amino acid residue W232 is critical for binding the COOH-terminal 20 amino acids of PEST-type PTP HSCF in vitro and in vivo. Cumulative evidence suggests that CD2BP1/PSTPIP1 and its interacting proteins are integral to actin reorganization during cytoskeletal-mediated events. Thus, the loss of the full complement of functional CD2BP1/PFP PEST may alter the context of the surface of activated T cells and possibly other lymphoid cells. Perhaps, as is proposed for other classic autoimmune diseases, an environmental agent triggers hyper-responsive T cell reaction to an as yet unidentified autoantigen present in skin and joints. The localized destruction of joints and the pyoderma gangrenosum in PAPA syndrome are clinical features associated with more complex autoimmune diseases, such as rheumatoid arthritis and inflammatory bowel disease, and suggests some etiological overlap with these disorders.

Methods

Yeast Two-hybrid Analysis. The wild type CD2BP1 cDNA was PCR amplified from a human spleen cDNA library using primer 1: ATT GGA TCC AGC TGC AGT TCA AAG ATG, SEQ ID NO:23; and primer 2: AAT GTC GAC TAG CAA GGT GGG GAC AGT GC, SEQ ID NO:24. The CD2BP1 SH3 domain deletion construct was built using primer 1 and primer 3: AAT GTC GAC GGG TTT CCC TGT ATC TCC TGT, SEQ ID NO:25. Amplification products were purified, restriction digested with Bam HI and Sal I, and cloned into the corresponding sites of pAS2-1 (Clontech). The E250Q and A230T mutant cDNA clones were derived by PCR site-directed mutagenesis of wild-type CD2BP1 and subcloned into pAS2-1. The cytoplasmic domain of CD2 was PCR amplified from a human spleen cDNA library using primer 4: ATT GGA TCC GAG CCC ACA GAG TAG CTA CTG A, SEQ ID NO:26; and primer 5: AAT GTC GAC TAG CAA GGT GGG GAC AGT GC, SEQ ID NO:27. Amplification products were purified, restriction digested with Bam HI and Sac I, and cloned into the corresponding sites of ACT2AD (Clontech). Wild-type and mutant constructs were purified, and the DNA sequence of the fusion proteins was confirmed by automated fluorescence sequencing. Yeast two-hybrid screening was performed using the strains, media, vectors, and protocols from the Matchmaker two-hybrid system (Clontech). Cells containing the wild-type CD2BP1 -GAL4BD construct were transformed with 10 ug of a human thymus cDNA library in pACT2 (Clontech).

Approximately 2×10⁵ transformants were spread on SD/-Leu/-Trp/-His/+3-AT plates. An aliquot was also plated on SD/-Leu/-Trp/ plates to test co-transformation efficiencies. After incubation at 30° C., positive clones were patched on SD/-Leu/-Trp/-His/+3-AT (25 mM) plates, and assayed for β-galactosidase (β-GAL) activity using a colony-lift filter assay. Quantitative β-GAL assays were performed in triplicate using CPRG (Roche Molecular Biochemicals) as a substrate. Binding domain constructs were cured from yeast clones by growth in -Leu media followed by duplicate plating to -Trp media to identify clones that had lost the GAL4BD construct. These cured clones were then re-transformed with either the E250Q or A230T mutant-GAL4BD constructs or with the wild type construct and were retested for histidine prototrophy.

All of the compositions and methods disclosed and claimed herein can be made and executed without undue experimentation in light of the present disclosure. While the compositions and methods of this invention have been described in terms of preferred embodiments, it will be apparent to those of skill in the art that variations maybe applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of diagnosing PAPA syndrome in a subject comprising detecting a mutation in a gene allele of the subject, wherein the gene allele encodes CD2BP1.

2. The method of claim 1, wherein the mutation is within the region of the gene that encodes from amino acid 122 to 288 inclusive in CD2BP1.

3. The method of claim 1, wherein the mutation includes a G748C transversion in a CD2BP1 gene.

4. The method of claim 1, wherein the mutation includes a G688A transition in a CD2BP1 gene.

5. The method of claim 1, wherein the detection is by denaturing HPLC.

6. The method of claim 1, wherein the detection is by DNA sequence analysis.

7. A method of diagnosing PAPA Syndrome in a subject comprising identifying a single nucleotide polymorphism (SNP) in the CD2BP1 gene of the subject, comprising:
(a) obtaining a sample of nucleic acid from the subject; and
(b) determining the identity of one or more SNPs in the CD2BP1 gene, wherein the SNPs are located at nucleotides 688 and 748 of the CD2BP1 gene.

8. The method of claim 7, wherein the determining step comprises amplifying at least a portion of a nucleic acid molecule encoding the CD2BP1 gene.

9. The method of claim 7, wherein the identity of one or more SNPs is determined by DNA sequence analysis.

10. A method of screening for an agent that modifies an immune response in cells that express a CD2BP1 with a mutation associated with PAPA syndrome, the method comprising:
contacting the cells with an agent suspected of modifying the immune response;
measuring an indicator of immune response; and
comparing the measurement to the same immune response indicator in a control cell under comparable conditions in the absence of the agent;
wherein a difference in the indicator is indicative of an agent that modifies the immune response.

11. The method of claim 10, wherein the mutation is in the region of CD2BP1 bounded by amino acids 122 to 288 inclusive.

12. The method of claim 10, wherein the cells express CD2BP1 with a G668A mutation as numbered in SEQ ID NO:21.

13. The method of claim 10, wherein the cells express CD2BP1 with a G748C mutation as numbered in SEQ ID NO:19.

14. The method of claim 10, wherein the mutation is in amino acid 250 of the CD2BP1.

15. The method of claim 10, wherein the mutation is an E250Q mutation of the CD2BP1.

16. The method of claim 10, wherein the mutation is in amino acid 230 of the CD2BP1.

17. The method of claim 10, wherein the mutation is an A230T mutation of the CD2BP1.

18. The method of claim 10, wherein the indicator is T-cell resetting.

19. The method of claim 10, wherein the indicator is altered motility of the cells.

20. The method of claim 10, wherein the indicator is CD2 triggered adhesion involving CD58.

21. The method of claim 10, wherein the indicator is integrin-mediated adhesion activated through CD2 or a CD15 carrier.

22. The method of claim 21, wherein the CD15 carrier is CD66a.

23. A method of screening for an agent that modifies an immune response in cells that express a mutant CD2BP1, wherein the mutant CD2BP1 comprises a A230T mutation or a E250Q mutation, comprising:
contacting the cells with an agent suspected of modifying the immune response;
measuring an indicator of immune response; and
comparing the measurement to the same immune response indicator in control cells under comparable conditions in the absence of the agent;
wherein a difference in the indicator is indicative of an agent that modifies the immune response.

24. The method of claim 23, wherein the indicator is T-cell rosetting.

25. The method of claim 23, wherein the indicator is altered motility of the cells.

26. The method of claim 23, wherein the indicator is CD2 triggered adhesion involving CD58.

27. The method of claim 23, wherein the indicator is integrin-mediated adhesion activated through CD2 or a CD15 carrier.

28. The method of claim 27, wherein the CD15 carrier is CD66a.

29. A method of screening for an agent that modifies an interaction of CD2BP1 with a biding partner of CD2BP1, wherein the CD2BP1 has a mutation associated with PAPA syndrome, the method comprising:
measuring the binding of the CD2BP1 to a binding partner in the presence of an agent suspected of altering the binding interaction of the CD2BP1 and the binding partner and comparing the binding in the presence of the agent to a measured control of the binding interaction in the absence of the agent, wherein a difference in the binding interaction is indicative of an effector of CD2BP1 binding to a binding partner.

30. The method of claim 29, wherein the binding partner is the cytoplasmic portion of CD2, PTP PEST, PTP HSCF, WASP, pyrin, c-Abl, or a CD15 carrier.

31. The method of claim 29, wherein the mutation is in the region of CD2BP1 bounded by amino acids 122 to 288 inclusive.

32. The method of claim 29, wherein the mutation is in amino acid 250 of the CD2BP1.

33. The method of claim 29, wherein the mutation is an E250Q mutation of the CD2BP1.

34. The method of claim 29, wherein the mutation is in amino acid 230 of the CD2BP1.

35. The method of claim 29, wherein the mutation is an A230T mutation of the CD2BP1.

36. A method of screening for an agent that modifies an interaction of mutant CD2BP1 with a binding partner of CD2BP1, wherein the mutant CD2BP1 comprises an A230T mutation or an E250Q mutation, the method comprising:
measuring the binding of the mutant CD2BP1 to a binding partner in the presence of an agent suspected of altering the binding interaction of the mutant CD2BP1 and the binding partner, and comparing the binding in the presence of the agent to a measured control of the binding interaction in the absence of the agent, wherein a difference in the binding interaction is indicative of an effector of mutant CD2BP1 binding to a binding partner.

37. The method of claim 36, wherein the binding partner is the cytoplasmic portion of CD2, PTP PEST, PTP HSCF, WASP, pyrin, c-Abl, or a CD15 carrier.

38. An isolated nucleic acid molecule or the complement thereof, wherein the molecule encodes an amino acid sequence comprising the sequence of SEQ ID NO:20 or SEQ ID NO:22, with conservative amino acid substitutions.

39. The molecule of claim 38, wherein the molecule encodes an amino acid sequence comprising the sequence of SEQ ID NO:20.

40. The molecule of claim 38, wherein the nucleic acid molecule comprises the nucleic acid sequence of SEQ ID NO:19.

41. The molecule of claim 38, wherein the molecule encodes an amino acid sequence comprising the sequence of SEQ ID NO:22.

42. The molecule of claim 38, wherein the nucleic acid molecule comprises the nucleic acid sequence of SEQ ID NO:21.

43. An expression construct comprising the nucleic acid molecule of claim 38 operably linked to an expression control sequence.

44. The expression construct of claim 43, further defined as a plasmid expression vector or a viral expression vector.

45. A host cell transformed or transfected with the expression construct of claim 43, or a progeny of the cell.

46. The host cell of claim 45, further defined as a bacterial cell, a mammalian cell, or a human cell.

47. An isolated nucleic acid molecule comprising about 20 contiguous nucleotides of SEQ ID NO:18, including: (a) nucleotide 688 wherein the G is replaced by an A; (b) nucleotide 748 wherein the G is replaced by a C; or both (a) and (b).

48. The isolated nucleic acid molecule of claim 47, wherein the nucleotide corresponding to nucleotide 688 of SEQ ID NO:18 is located at the 5′ end of the molecule.

49. The isolated nucleic acid molecule of claim 47, wherein the nucleotide corresponding to nucleotide 688 of SEQ ID NO:18 is located at the 3′ end of the molecule.

50. The isolated nucleic acid molecule of claim 47, wherein the nucleotide corresponding to nucleotide 748 of SEQ ID NO:18 is located at the 5′ end of the molecule.

51. The isolated nucleic acid molecule of claim 47, wherein the nucleotide corresponding to nucleotide 748 of SEQ ID NO:18 is located at the 3′ end of the molecule.

52. An isolated nucleic acid molecule comprising the complement of the nucleic acid molecule of claim 47.

53. An array of nucleic acid molecules attached to a solid support, the array comprising an oligonucleotide that will hybridize to the nucleic acid molecule of claim 47, under conditions in which the oligonucleotide will not substantially hybridize to a nucleic acid molecule consisting of SEQ ID NO:18.
